(19) United States
(12) Patent Application Publication
(10) Pub. No.: US 2020/0113511 A1
(43) Pub. Date: Apr. 16, 2020

(54) METHOD AND APPARATUS FOR DYNAMIC DIAGNOSIS OF MUSCULOSKELETAL CONDITIONS

(71) Applicants: Lanny Leo Johnson, Frankfort, MI (US); Gerald R. Williams, JR., Villanova, PA (US); David DiPaola, North Potomac, MD (US); James R. Andrew, Frankfort, MI (US)

(72) Inventors: Lanny Leo Johnson, Frankfort, MI (US); Gerald R. Williams, JR., Villanova, PA (US); David DiPaola, North Potomac, MD (US); James R. Andrew, Frankfort, MI (US)

(21) Appl. No.: 16/600,218

(22) Filed: Oct. 11, 2019

Related U.S. Application Data
(60) Provisional application No. 62/745,075, filed on Oct. 12, 2018.

(51) Int. Cl. A61B 5/00; A61B 5/11

(52) U.S. Cl. CPC A61B 5/4538; A61B 2562/028; A61B 5/11; A61B 5/0002

(57) ABSTRACT

The disclosure is directed to a system and methods for dynamic diagnosis of musculoskeletal conditions. The methods include monitoring at least one body part of a subject with at least one sensor, the at least one sensor configured to detect motions of the at least one body part of the subject, the at least one sensor comprising a Micro Electro Mechanical System (MEMS) sensor. The at least one sensor transmits kinematic data to a signature-comparing device. The methods include obtaining composite signatures of the subject based on the kinematic data, the composite signatures comprising at least one motion signature. The methods also include comparing the composite signatures of the subject to a normal composite signature to determine whether a difference between the composite signatures of the subject and the normal composite signatures is larger than a pre-determined threshold.

### RELATED APPLICATION

This application claims priority to U.S. Provisional Application No. 62/745,075, filed with the United States Patent and Trademark Office on Oct. 12, 2018, which is incorporated by reference in its entirety.

### BACKGROUND

### 1. Technical Field

The present disclosure relates to a system for and methods of making dynamic diagnosis of musculoskeletal conditions. In particular, the present disclosure relates to methods and a system to provide dynamic diagnosis based on analyzing motion signatures of a subject's movement.

### 2. Background Information

There is no dynamic means of making a specific, sensitive, reliable musculoskeletal diagnosis of musculoskeletal conditions. Conventional methods of diagnosing musculoskeletal and psychomotor conditions rely upon medical history and physical examination of the subject, laboratory testing, plain film radiographs, ultrasound, and magnetic resonance imaging (MRI). Thus, the existing method of diagnosing musculoskeletal and psychomotor conditions is time consuming, expensive, and not always reliable. Moreover, conventional techniques are not dynamic, but typically static, and the data collected by such methods do not provide a dynamic diagnosis of musculoskeletal conditions in a single test based on patient musculoskeletal signatures for a specific diagnosis.

The present disclosure is directed toward addressing one or more drawbacks, including but not limited to those set forth above. The present disclosure improves the medical diagnosis of musculoskeletal conditions and provides a more accurate, more sensitive, specific, reliable and reproducible means in applications of clinical impression, surgical indications, and clinical investigation.

### BRIEF SUMMARY

The present disclosure describes a method for dynamically diagnosing musculoskeletal conditions related to at least one body part of a subject. The method includes monitoring at least one body part of a subject with at least one sensor, the at least one sensor configured to detect motions of the at least one body part of the subject, the at least one sensor comprising a Micro Electro Mechanical System (MEMS) sensor. The method includes transmitting kinematic data captured when the subject moves the at least one body part in a pre-determined kinematic pattern from the at least one sensor to a signature-comparing device, the kinematic data corresponding to the motions of the at least one body part when the subject moves the at least one body part in the pre-determined kinematic pattern. The method includes obtaining composite signatures of the subject based on the kinematic data, the composite signatures comprising at least one motion signature. The method includes comparing the composite signatures of the subject to a normal or pre-determined established standard composite signature to determine whether a difference between the composite signatures of the subject and the normal composite signatures is larger than a pre-determined threshold. The method includes diagnosing, in response to determining that the difference between the composite signatures of the subject and the normal composite signatures is larger than a pre-determined threshold, the subject as having a specific musculoskeletal condition.

The present disclosure describes a system for dynamic diagnosing musculoskeletal conditions related to at least one body part of a subject in response to a set of instructions for instructing the subject to move the at least one body part in a pre-determined kinematic pattern. The system includes at least one sensor attached to the at least one body part of the subject, the at least one sensor configured to detect motions of the at least one body part of the subject. The system includes a signature-comparing device receiving kinematic data transmitted from the at least one sensor, the kinematic data corresponding to the motions of the at least one body part when the subject is instructed to move the at least one body part in the pre-determined kinematic pattern, so that the signature-comparing device obtains composite signatures of the subject based on the kinematic data, the composite signatures comprising at least one motion signature, compares the composite signatures of the subject to normal composite signatures to determine whether a difference between the composite signatures of the subject and the normal composite signatures is larger than a pre-determined threshold, and diagnoses, in response to determining that the difference between the composite signatures of the subject and the normal composite signatures is larger than a pre-determined threshold, the subject as having a specific musculoskeletal condition.

The present disclosure also describes a system for dynamic diagnosing musculoskeletal conditions related to at least one body part of a subject. The system includes at least one sensor configured to detect motions of at least one body part of a subject. The at least one sensor is attached to the at least one body part of a subject, and the subject, in response to a set of instructions, is instructed to move the at least one body part in a pre-determined kinematic pattern. When the subject moves the at least one body part in the pre-determined kinematic pattern, the at least one sensor transmits kinematic data corresponding to the motion of the at least one body part to a signature-comparing device. The system includes a signature generating circuitry in the signature-comparing device generating composite signatures based on the kinematic data. The system includes a comparison circuitry in the signature-comparing device comparing the composite signatures and normal composite signatures to determine whether a difference between the composite signatures of the subject and the normal composite signatures is larger than a pre-determined threshold. The system includes a diagnosing circuitry providing result of the subject as having a specific musculoskeletal condition in response to determining that the difference between the composite signatures of the subject and the normal composite signatures is larger than a pre-determined threshold.

### BRIEF DESCRIPTION OF THE DRAWINGS

FIG. 1 is a schematic diagram of a dynamic diagnosis system for musculoskeletal conditions.

FIG. 2 is a schematic diagram of a sensor.

FIG. 3 is a flow diagram of a sensor wherein flow diagram of a method for a system dynamic diagnosis of musculoskeletal conditions.

FIG. 4 is a flow diagram of the step 330 as shown in FIG. 3.

FIG. 5 is a schematic diagram of a location of the system.

FIG. 6A is a schematic diagram of an arm motion of a subject performing the first test of forward extension.

FIG. 6B is a schematic diagram of a Scapular plane shown in a top view from a human subject.

FIG. 7 is a schematic diagram of an arm motion of a subject performing the second test of external rotation.

FIG. 8 is a schematic diagram of an orientation of an accelerometer.

FIG. 9 is a schematic diagram of an orientation of a gyroscope.

FIG. 10 is a schematic diagram of a synchronization between the sensor system and video.

FIG. 11 is a schematic diagram of an acceleration signature along x-axis and its corresponding threshold.

FIG. 12 is a schematic diagram of a rotation signature along x-axis and its corresponding threshold.

FIG. 13 shows an exemplary embodiment where motion signatures from normal subjects are obtained during forward extension tests without weight.

FIG. 14 shows an exemplary embodiment where motion signatures from normal subjects are obtained during forward extension tests with weight.

FIG. 15 shows an exemplary embodiment where motion signatures from normal subjects are obtained during external rotation tests without weight.

FIG. 16 shows an exemplary embodiment where motion signatures from normal subjects are obtained during external rotation tests with weight.

FIG. 17 shows an exemplary embodiment where motion signatures from an abnormal subject are obtained.

FIG. 18 shows another exemplary embodiment where motion signatures from an abnormal subject are obtained.

FIG. 19 shows an exemplary embodiment where motion signatures from an abnormal subject without weight are obtained.

FIG. 20 shows an exemplary embodiment where motion signatures from an abnormal subject with weight are obtained.

FIG. 21 is a flow diagram of one embodiment for a method for diagnosing musculoskeletal conditions of ulnar collateral ligament (UCL).

FIG. 22A is a schematic diagram of a location of a Micro Electro Mechanical System (MEMS) sensor.

FIG. 22B is another schematic diagram of the location of the MEMS sensor shown in FIG. 22A.

FIG. 23A is a schematic diagram of a subject's position and/or motion performing a test for diagnosing musculoskeletal conditions of UCL.

FIG. 23B is another schematic diagram of a subject's position and/or motion performing a test for diagnosing musculoskeletal conditions of UCL.

FIG. 23C is another schematic diagram of a subject's position and/or motion performing a test for diagnosing musculoskeletal conditions of UCL.

FIG. 24 is an exemplary embodiment where motion signatures from a normal subject are obtained during a test for diagnosing musculoskeletal conditions of UCL.

FIG. 25 is an exemplary embodiment where motion signatures from an abnormal subject is obtained during a test for diagnosing musculoskeletal conditions of UCL.

FIG. 26 is an exemplary embodiment where motion signatures from another abnormal subject is obtained during a test for diagnosing musculoskeletal conditions of UCL.

### DETAILED DESCRIPTION OF THE DISCLOSURE

The disclosed systems and methods will now be described in detail hereinafter with reference to the accompanied drawings, which form a part of the present application and show, by way of illustration, specific examples of embodiments. The systems and methods may, however, be embodied in a variety of different forms and, therefore, the covered or claimed subject matter is intended to be construed as not being limited to any of the embodiments to be set forth below. The disclosure may be embodied as methods, devices, components, or systems. Accordingly, embodiments of the disclosed system and methods may, for example, take the form of hardware, software, firmware or any combination thereof.

Throughout the specification and claims, terms may have nuanced meanings suggested or implied in context beyond an explicitly stated meaning. Likewise, the phrase “in one embodiment” or “in some embodiments” as used herein does not necessarily refer to the same embodiment and the phrase “in another embodiment” or “in other embodiments” as used herein does not necessarily refer to a different embodiment. Similarly, the phrase “in one implementation” or “in some implementations” as used herein does not necessarily refer to the same implementation and the phrase “in another implementation” or “in other implementations” as used herein does not necessarily refer to a different implementation. It is intended, for example, that claimed subject matter may include combinations of exemplary embodiments or implementations in whole or in part.

In general, terminology may be understood at least in part from usage in context. For example, terms, such as “and”, “or”, or “and/or,” as used herein may include a variety of meanings that may depend at least in part upon the context in which such terms are used. In addition, the term “one or more” or “at least one” as used herein, depending at least in part upon context, may be used to describe any feature, structure, or characteristic in a singular sense or may be used to describe combinations of features, structures or characteristics in a plural sense. Similarly, terms, such as “a”, “an”, or “the”, again, may be understood to convey a singular usage or to convey a plural usage, depending at least in part upon context. In addition, the term “based on” or “determined by” may be understood as not necessarily intended to convey an exclusive set of factors and may, instead, allow for existence of additional factors not necessarily expressly described, again, depending at least in part on context.

The present disclosure describes a method and a system for dynamic diagnosis of musculoskeletal conditions. The musculoskeletal tissue may include, but not be limited to, a body part, a muscle, a tendon, a ligament, or a joint contents or combination of those anatomical entities. The musculoskeletal conditions may include, but not be limited to, instability of a musculoskeletal tissue structure or joint, competency of a musculoskeletal tissue structure or joint, and deficiencies of a musculoskeletal tissue structure or joint. The musculoskeletal structure, joint, and/or ligament may include, but not be limited to, head, neck, shoulder, arm, elbow, forearm, wrist, hand and fingers, the spine, pelvis, hip, thigh, knee, leg, ankle, or foot and toes. The present disclosure improves the medical diagnosis of musculoskeletal conditions and has utility in detecting joint, muscle, tendon, and even neuromuscular deficiencies in forming clinical impression, surgical indications, and clinical investigation and research.

The present disclosure may enable an early diagnosis and/or profile those who are susceptible to loosening and/or infection. Infected and/or loose total joints may cause major humanitarian and societal costs. They may be difficult to diagnose early and/or even late. The present disclosure describes a method/system with a Micro Electro Mechanical System (MEMS) technology may profile patients with these implants that are known not to be loose with a profile and compare to those know to be loose and/or infected, or both.

The present disclosure may enable a physician to monitor recovery and rehabilitation progress of a patient remotely. The monitoring of the patient may be through an online cloud with a common hardware, for example, a smart watch or cell phone with a developed APP. The remote monitoring may save both time and costs as the remote monitoring may be less expensive than a full doctor visit. The physician may check in with the patient via face time if needed.

The present disclosure may provide a physician quantifiable data to measure the success of medical interventions, with either new procedures or old procedures, so that the best technique may be identified and deployed in future treatment.

The present disclosure may be used to monitor the results of physical therapy enabling therapists to use the best techniques and change routines if progress is not being made.

The present disclosure may be used to identify subjects with unresolved medicolegal issues who may be faking an injury in order to obtain a specific benefit, for example and not limited to, workers compensation. The subject may perform the motion tests described in this present disclosure, and the sensitivity, specificity, and repeatability of the described system may make it difficult to manipulate the result.

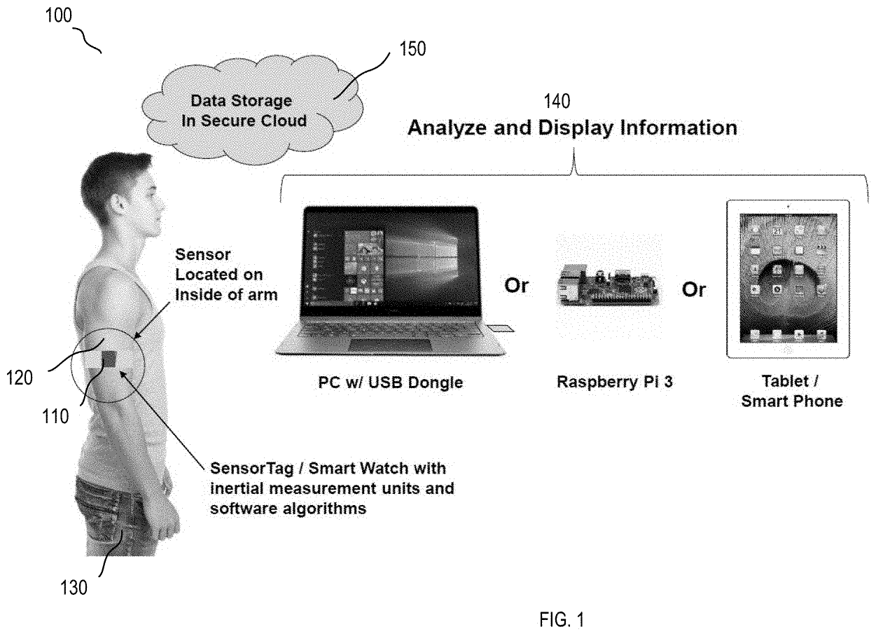

A system 100 is shown in FIG. 1 for dynamic diagnosis of musculoskeletal conditions. The system 100 may include a sensor or a set of sensors 110. A sensor attached to a body part 120 of a subject 130 may generate/sense/measure kinematic data of the body part of the subject.

The subject 130 may be a human being or an animal. For the disclosure below, the human being is used as an example to describe the disclosure and does not impose any limitation to the present disclosure. The body part of the human being may include head, neck, shoulder, arm, elbow, forearm, wrist, hand and fingers, the spine, pelvis, hip, thigh, knee, leg, ankle, or foot and toes, etc.

The sensor may transmit the measured kinematic data to one or more electronic devices 140 for further analyzing and processing. The data transmission may be either a wired transmission or a wireless transmission. The wireless transmission may include, for example and not limited to, Bluetooth, Bluetooth Low Energy (BLE), Zigbee, Z-Wave, 6LoWPAN, WI-FI or other wireless technology. The wireless communication may take place via radio frequency similar to a radio, or ultrasound. The wireless communication may enable connection to cellular network via a smart phone or a computer enabled with WI-FI. Wireless sensing may be preferred to allow free movement of limbs and body. Short range wireless such as Bluetooth and Bluetooth Low Energy (BLE) provides excellent battery life.

The one or more electronic devices 140 may include, for example and not limited to, a smart phone, a computer/laptop, a Raspberry Pi 3, or a tablet. The one or more electronic devices may receive the kinematic data from the sensor 110. The one or more electronic devices 140 may analyze and process the kinematic data using at least one algorithm and then report the results. The kinematic data may be multi-dimensional data, for example but not limited to, three spatial dimensions and one temporal (time) dimension. In one implementation, the results may be displayed in graphical form for the physician along with a suggestion for normal or abnormal classification. Abnormal classification may indicate an injury requiring surgical repair.

The sensor 110 may transmit the measured kinematic data to an external data storage device 150 for data storage and/or further data processing. The data transmission may be either a wired transmission or a wireless transmission. The external data storage device 150 may include an on-site data server, which may be in the same room or in the same building as the location of the sensor 110 and the human being. In another implementation, the external data storage device 150 may include an off-site on-line data storage device, for example and not limited to, a data cloud.

In one implementation, the sensor 110 may be an accelerometer, so that the sensor 110 attached to a body part 120 may measure an acceleration of the body part 120. When acceleration data from the accelerometer is analyzed as a function of time, a speed of the body part 120 may be calculated given a known speed at a known time point. For example, the known speed of the body part at the known time point may be zero when the body part is in a resting state at the time point of zero.

The acceleration data from the accelerometer may be analyzed to provide speed data or position data. Integration of the acceleration data with respect to time can provide speed data for the accelerometer. Integration of the speed data with respect to time can provide position data for the accelerometer.

In another implementation, the sensor 110 may be a gyroscope, so that the gyroscope attached to a body part may measure rotational angles of the body part. The rotational angles may be represented by an x-axis rotational angle, a y-axis rotational angle, and a z-axis rotational angle. When rotational angle data from the gyroscope is analyzed as a function of time, a rotational speed of the body part may be calculated since the rotational speed is a time derivative of the rotational angle. Similarly, a rotational acceleration may be calculated as well since the rotational acceleration is a second-order time derivative of the rotational angle.

In another implementation, the sensor 110 may be a magnetometer, so that the magnetometer attached to a body part may measure direction/orientation of the body part. The direction/orientation of the body part may be represented by an angle relative to one particular direction, e.g., the north direction.

The sensor 110 may be a set of sensors including one or more accelerometer, one or more gyroscope, or one or more magnetometer. When the set of sensors is attached to a body part, acceleration data, rotational angle data, and/or orientation data of the body part may be generated.

The sensor 110 may include a Micro Electro Mechanical System (MEMS) sensor. The MEMS sensor may be used to measure motion or locomotion of humans and animals. Depending on specific pathological conditions of the body part 120, the MEMS sensor may be properly assembled and tailored to the specific pathological conditions to provide a real time functional diagnosis not possible by any other previously existing means. In one implementation, an elbow joint, including the medial collateral ligament testing may be added. A composite signature combining one or more areas of the body will be used to assess a common function of a throwing motion that then would include combination of separate interrelated parts of the extremity and or body part; i.e. the throwing motion of an athlete may include assessing the shoulder, arm, elbow, forearm, wrist and hand.

The composite signature may include one or more signatures, which may be unique by demographics, by gender, and by disease or injury. A signature may differ by individuals as well. In one implementation, a signature of the composite signature may be similar in graphic to one produced by an electrocardiogram (ECG or EKG) for heart disease: for example, a EKG graph may be dynamic and have various patterns indicative of specific pathological conditions; and the signature of the composite signatures may be indicative of specific musculoskeletal conditions.

In a typical and conventional pathological diagnosis, a gross and/or a subsequent microscopic histological analysis may be static and include static findings. Being different, measurements with MEMS sensors may be dynamic and may produce a dynamic finding. In one implementation, the findings produced by MEMS measurements may include a dynamic feature that is similar to the EKG in graphics. In another implementation, the findings produced by MEMS measurements may be similar to pathologic microscopic findings because the results are highly sensitives and specific. For example but not limited to, a muscle of a unknown subject may be weak, and the initiation of motion of the muscle may have a slight delay, which may suggest that shaking vibrations may occur when weakness is demonstrated by applying weights to load the extremity. In another implementation, the findings produced by MEMS measurements may show the integrity or lack thereof of a musculotendinous group when one is torn or stretched, for example, the range of motion of the arc would be greater in the starting or finishing position.

MEMS sensors may be better than the human eye or other clinical examination in dynamically diagnosing musculoskeletal conditions. In one implementation, MEMS sensors as well as the developed software developer may produce signatures for diagnosing musculoskeletal conditions, while an expert shoulder surgeon may not find any supraspinatus abnormality.

MEMS sensors may be better than video in motion measurements. For example, video may be mostly recorded as sequences of two-dimensional images, and MEMS sensors may record motions in three-dimensional space with higher sensitivity.

The sensor 110 may be used to measure composite motion of any muscle, tendon, and/or joint under any circumstances in free space. The exemplary implementations/embodiments described in this application do not restrict measurement of the motion to a specific muscle, tendon, and or joint function. For example, MEMS sensors may be used to measure the position and motion of the human torso in activities of daily living.

In one embodiment, the sensor 110 may include a set of individual sensors. The set of individual sensors may include a combination of miniature sensors. MEMS sensors may include components between 1 and 100 micrometers in size (e.g., 0.001 to 0.1 mm), and MEMS devices generally range in size from 20 micrometers to a millimeter (e.g., 0.02 to 1.0 mm).

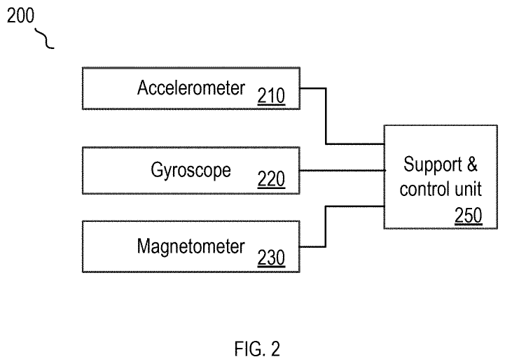

FIG. 2 shows an exemplary implementation of a sensor 200. The sensor 200 includes a 3-axis accelerometer 210, a 3-axis gyroscope 220, and a 3-axis magnetometer 230. The sensor 200 may include other supporting and/or control components (e.g. support & control unit 250), which may include but are not limited to, a microprocessor, memory, a wireless antenna, and a rechargeable battery. In one implementation, the sensor 200 may include commercially available products and be purchased off the shelf, so as to be relatively inexpensive.

In one implementation, in addition to obtaining kinematic data, the sensor 200 may analyze and process the obtained kinematic data to generate at least one motion signature. For example, the accelerometer 210 may obtain acceleration data as a function of time. The acceleration data may be transmitted to the support & control unit 250. The support & control unit 250 may analyze and process the acceleration data to generate motion signatures for accelerations, speeds, and/or positions of the sensor 200. In a similar manner the support & control unit 250 may analyze and process data from the gyroscope 220 and the magnetometer 230 to generate motion signatures for angular position, angular acceleration, angular velocity, orientation, change in orientation per unit time, and rate of change in orientation per unit time.

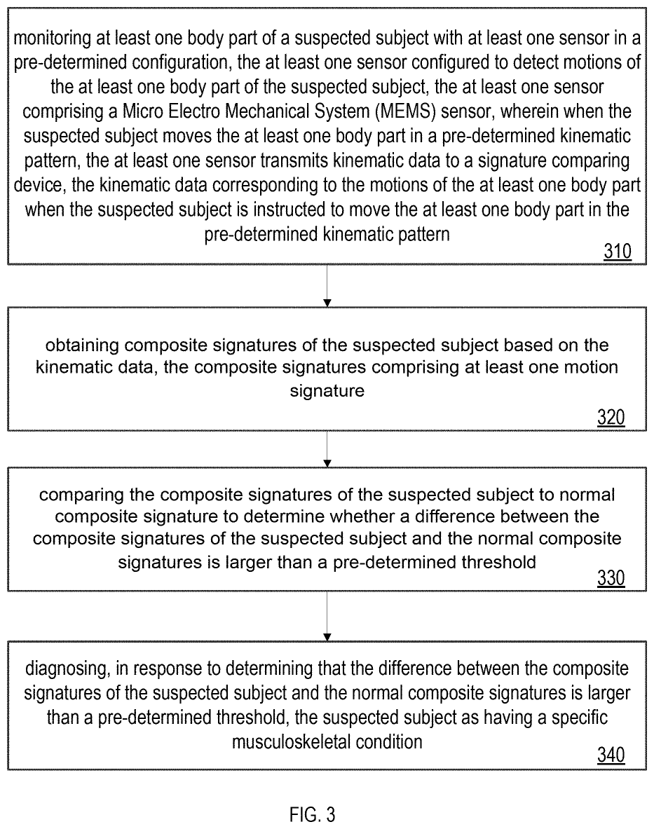

As shown in FIG. 3, the present disclosure describes a method for dynamically diagnosing musculoskeletal conditions related to at least one body part of a subject. The method may include the following steps.

Step 310 may include monitoring at least one body part of a subject with at least one sensor in a pre-determined configuration. The at least one sensor is configured to detect motions of the at least one body part of the subject. The at least one sensor may include a Micro Electro Mechanical System (MEMS) sensor. When the subject moves the at least one body part in a pre-determined kinematic pattern, the at least one sensor may transmit kinematic data to a signature-comparing device. The kinematic data may correspond to the motions of the at least one body part when the subject is instructed to move the at least one body part in the pre-determined kinematic pattern.

The subject is instructed to move at least one body part in the pre-determined kinematic pattern. The kinematic pattern may be pre-determined depending on the at least one body part or depending on a type of suspected musculoskeletal condition. For example, for a suspected supraspinatus injury within the shoulder, the kinematic pattern may include a motion of an arm. For another example, for suspected knee condition, the kinematic pattern may be pre-determined to include 1) bending the subject's knee with the leg hanging in passive flexion, 2) extending the knee by lifting the foot into extension, and 3) returning to the resting position. In one implementation, the subject may be a patient suspected to have certain musculoskeletal conditions. In another implementation, the subject may be a normal subject for testing purpose.

Each sensor may include a number of individual sensors, for example and not limited to, an accelerometer, a gyroscope, and/or a magnetometer. In one implementation, there may be one sensor. In another implementation, there may be multiple sensors, such as two, three, five, or ten sensors. The multiple sensors may include the same type of individual sensors, or each sensor of the multiple sensors may include different types of individual sensors.

The sensor may be attached to a particular location of the body part of the subject. For example, the sensor may be attached to a bicep or triceps of an inner arm of the subject.

When there are more than one sensor, the sensors may be attached to at least one body part of the subject in a particular configuration. For example but not limited to, when there are three sensors, a first sensor may be attached to a middle of an upper arm, a second sensor may be attached to an elbow, and a third sensor may be attached to a middle of a forearm.

The transmission of the kinematic data from the at least one sensor to the signature-comparing device may include wired and/or wireless transmission.

Step 320 may include obtaining composite signatures of the subject based on the kinematic data. The composite signatures includes at least one motion signature. Based on the kinematic data, at least one motion signature may be obtained. For example, when the kinematic data measured from an accelerometer includes acceleration data, the motion signature may include an acceleration signature, which includes a range of the acceleration values. The motion signature may include a highest acceleration value and a lowest acceleration value.

As described above, speed data may be obtained based on the acceleration data. Thus, when the kinematic data includes the acceleration data, the motion signature may include a speed signature, which includes a range of speed values. The motion signature may include a highest speed value and a lowest speed value. Similarly, position data may be obtained based on the acceleration data. Thus, when the kinematic data includes the acceleration data, the motion signature may include a position signature, which includes a range of the position values.

The composite signatures may include at least one motion signature, which unravel the complexity of the motion. For example, the composite signature may include at least one motion signatures based on kinematic data from an accelerometer, such as an acceleration signature, a speed signature, and/or a position signature.

In one embodiment, a starting time point of the kinematic data may be registered for each repeat of performing the pre-determined kinematic pattern by a same subject. The registered starting time point may be different for each repeat by the same subject and may be used to align the kinematic data when the kinematic data for all repeats by the same subject is grouped together.

In another embodiment, a starting time point of the kinematic data may be registered for each subject when the each subject performs the pre-determined kinematic pattern. The registered starting time point for each subject may be different and used to align the kinematic data when the kinematic data for all subjects is grouped together.

In another implementation, the composite signature may include at least one motion signature based on kinematic data from multiple sensors. For example, the multiple sensors may include an accelerometer and a gyroscope, and thus the composite signature may include a speed signature and an angular speed signature.

Step 330 may include comparing the composite signatures of the subject to normal composite signature to determine whether a difference between the composite signatures of the subject and the normal composite signatures is larger than a pre-determined threshold. The method may further include obtaining the normal composite signatures from a server by the signature-comparing device.

The normal composite signatures may include at least one normal motion signature including an acceleration signature, a speed signature, a position signature, an angular acceleration signature, an angular speed signature, an angular signature, a plane signature, and/or a timing signature.

Optionally, the plane signature may be obtained based on the position signature and/or an angular signature. The plane signature may be a motion signature to quantify how well the motion of the subject is within a plane. For example, the plane may include a Scapular plane.

Optionally, the timing signature may be a motion signature to qualify the time durations between two pre-determined events. For example, a first pre-determined event may include a first motion when a raising arm reaches a horizontal position; and second pre-determined event may include a second motion when the raising arm reaches a highest position. The timing signature may be based on the time duration between the first pre-determined event and the second pre-determined event.

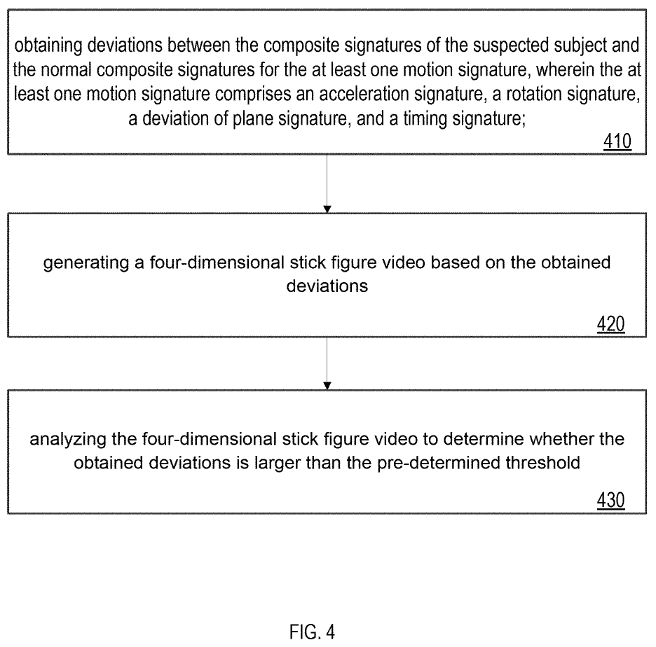

As shown in FIG. 4, step 330 may optionally include steps 410, 420, and 430. Step 410 may include obtaining deviations between the composite signatures of the subject and the normal composite signatures for the at least one motion signature. The at least one motion signature comprises an acceleration signature, a rotation signature, a deviation of a plane signature, and a timing signature. In one implementation, the deviation of the plane signature may be the difference between abnormal and normal signatures, for example and not limited to, an arm may move outside a scapular plane and may be represented more clearly with a three-dimensional or four-dimensional video.

Step 420 may include generating a four-dimensional stick figure video based on the obtained deviations as a means of further illustrating the results. The video's four dimensions may include x, y, z, and various motion signatures.

A video may be generated from the accelerometer, gyroscope and compass data. The video may be a four-dimensional stick figure video, including three spatial dimensions and one temporal (time) dimension. The video may display the data in 3D animated form to aid in diagnostics. In one implementation, the video may be used for threshold analysis when the data is projected as a hologram, for example, using the HoloLens from Microsoft (https://www.microsoft.com/en-US/hololens) or other competing manufacturers. When a hologram is used, a red blinking light may indicate a person in the hologram has reached/passed a threshold. A visual representation of the data in human animation may provide insight otherwise missed in looking at charts.

Step 430 may include additional analyzing the four-dimensional stick figure video by way of extended visualization to determine whether the obtained deviations is larger than the pre-determined threshold.

Step 340 may include diagnosing, in response to determining that the difference between the composite signatures of the subject and the normal composite signatures is larger than a pre-determined threshold, the subject as having a specific musculoskeletal condition.

### Embodiment for Dynamic Diagnosis of Shoulder Conditions

The present disclosure describes an embodiment of a system for dynamic diagnosis of shoulder conditions. The shoulder conditions may include abnormal conditions, for example but not limited to, a shoulder injury of a supraspinatus tear.

The system includes a sensor to measure locomotion of a body part of a subject. The sensor may be attached to a particular location relative to the body part. The attachment may be achieved by adhesive, rubber band, clips, or clamps. The relative location and orientation of the sensor may keep constant relative to the body part during the movement of the body part.

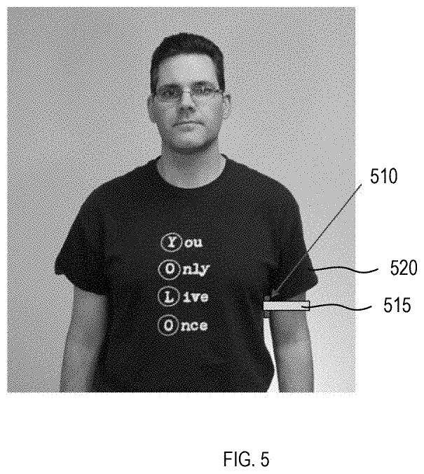

As FIG. 5 shows, the sensor 510 may fit in the recess between the bicep and triceps of the inner arm of an arm 520. The sensor 510 may be attached to the arm 520 by a securing band 515. A portion of a housing of the sensor 510 may have a triangular shape so that it may fit in the recess between the bicep and triceps of the arm 520.

The sensor 520 may include a MEMS accelerometer, which may measure the acceleration of gravity in three axes to determine position of the arm from a known reference. The sensor 520 may include a MEMS gyroscope, which may measure the rotational angle to obtain the angular velocity. Optionally, the sensor 520 may include a magnetometer, which generates magnetometer data.

The hardware to make these measurements could include a TI SensorTag, Apple Watch or any other off-the-shelf or custom device having a MEMS accelerometer and gyroscope with sufficient performance and within a short range of the wireless communications.

For each diagnosis, the corresponding tests may be pre-determined to include specific motion patterns.

For example, when a supraspinatus injury is suspected, elevation in the scapular plane and external rotation with the arm at the side may be the most affected motion patterns. For elevation in the scapular plane, the tested motion pattern may include a starting position with the upper extremity hanging at the subject's side with the elbow extended, elevation to a maximal overhead position, and return to the starting position. For external rotation with the arm at the side, the tested motion pattern may include a starting position with the patient's palm of the hand resting on the umbilicus, external rotation to a maximally externally rotated position, and return to the starting position.

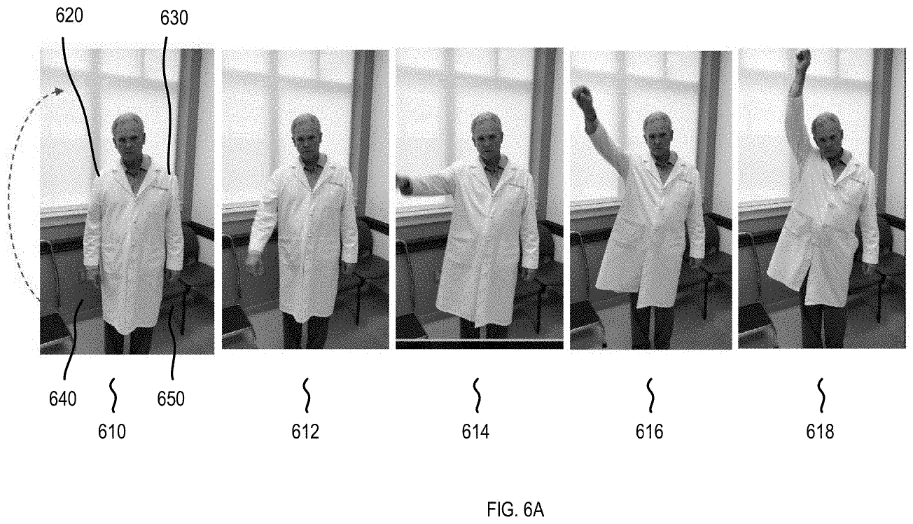

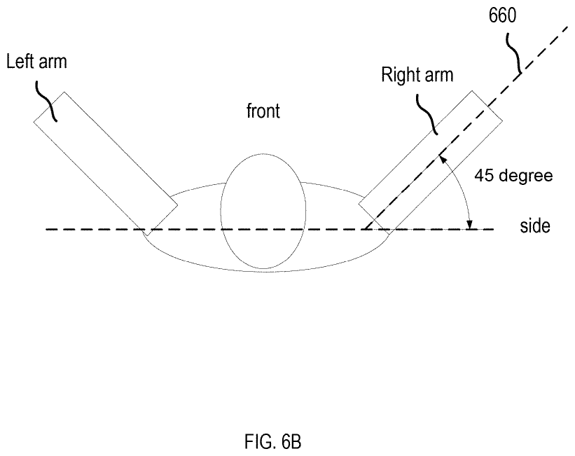

For another example, when infraspinatus injury is suspected, external rotation with the arm elevated may be the most affected motion pattern. The tested motion pattern for infraspinatus injury may include a starting position with the arm at the subject's side and the palm facing the thigh, elevation and external rotation to a maximum position of the palm against the back of the stationary head, and a return to the starting position. This motion pattern favors infraspinatus function over supraspinatus function based upon the known anatomical attachment sites and lines of action of the two muscles. This motion pattern may be used in addition to the previously described motion patterns for supraspinatus. In one implementation, the tested motion pattern may be a motion pattern as illustrated in FIG. 6A.

For another example, when subscapularis injury is suspected, internal rotation may be the most affected motion pattern. The tested motion pattern for subscapularis injury may include a starting position with the palm of the hand against the umbilicus and the humerus against the body, rotation of the humerus forward to a maximally internally rotated position while the hand stays against the umbilicus, and a return to the starting position. This may be done in a standing or supine position. This motion pattern may be done with all previously described motion patterns. All motion pattern may be performed with and without a weight grasps by the subject's hand. These combinations of motion patterns, with and without weights will elucidate specific motion signatures of combined injury patterns.

For another example, when inferior subluxation/dislocation of the shoulder or injury to the rotator interval capsule is suspected, inferior translation of the humerus with the arm at the side may be the most affected motion pattern. The tested motion pattern may include a starting position of the subject standing with the arm hanging to the side and the palm facing the thigh, the subject being handed a series of weights and being asked to hold the weight while keeping the muscles around their shoulder as relaxed as possible, and then releasing the weight. The test may be repeated with the palm facing forward and backward (i.e. anteriorly and posteriorly). This testing pattern highlights rotator interval function in internal, neutral, and external rotation. The sensors are on the arm and may include a second sensor placed adjacent to the shoulder; i.e. scapula or spine.

For another example, when anterior subluxation is suspected, abduction, extension, and external rotation with the arm elevated may be the most affected motion patterns. The tested motion pattern may include, while the subject is sitting or standing with or without weight in hand, taking the arm into abduction in plane of scapula and 90 degrees elevation, moving arm into maximal external rotation and returning to starting position. Another tested motion pattern may include a starting position of the supine subject with the palm of the hand on the back of the head and the elbow facing the ceiling, moving the elbow to a maximum posterior position while keeping the palm on the back of the head, and returning to the starting position.

For another example, when posterior subluxation is suspected, the motion pattern of the tests may include, while the subject is prone on floor with monitor on the arm, performing a standard push up and back down. Another tested motion pattern may include a starting position of the supine subject with the arm extended straight out to the side, moving the arm across the body with the palm on the posterolateral aspect of the opposite shoulder, and returning to the starting position.

There may be two tests for the subject to perform: a first test is a forward extension and a second test is an external rotation. During each test, the subject may be asked to repeat the motion for a number of times with or without a weight in the hand of the subject. Tests with weight may be used to enhance the detection of musculoskeletal conditions due to added stress during the test.

The number of times may be 1, 2, 3, 7, 10, or any integer number. The weight may be 1 lb, 2 lbs, 5 lbs, 8 lbs, 10 lbs, 20 lbs, or any number of pounds.

FIG. 6A shows an arm motion of a subject performing the first test of forward extension, where a sensor may be attached to a right arm of a right shoulder 620 of the subject. During the forward extension test, the subject may be instructed to put both arms at the subject's sides (e.g., right side 640 and left side 650) in 610 and elevate the right arm in a Scapular plane in 612. The subject may keep elevating the right arm 180 degree overhead in 614, 616, and 618. Then the subject may back down at the side. The Scapular plane 660 shown in FIG. 6B is a plane at 45 degree between front and side positions. FIG. 6A shows the forward extension test of the right shoulder 620 without weight. Alternatively, the subject may hold a weight in the right hand and perform the same motion as described above to perform the forward extension test of the right shoulder 620 with weight.

In another implementation, the subject may have a sensor attached to a left arm of a left shoulder 630 and perform the corresponding motion as described above to perform the forward extension test of the left shoulder 630 without weight. Alternatively, the subject may hold a weight in the left hand and perform the corresponding motion to perform the forward extension test of the left shoulder 630 with weight.

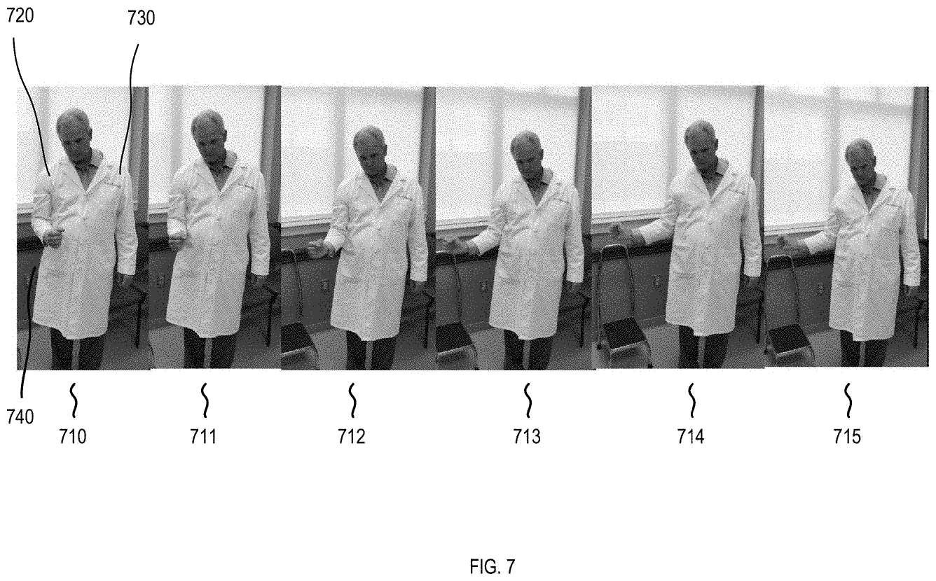

FIG. 7 shows an arm motion of a subject performing the second test of external rotation, where a sensor may be attached to a right arm of a right shoulder 720 of the subject. During the external rotation test, the subject may be instructed to position the right arm 720 in an “L” orientation by the right side 740 in 710. The subject may rotate the right arm 720 from front to back in 711, 712, 713, 714, and 715 sequentially. Then the subject may rotate the right arm 720 from back to front. FIG. 7 shows the external rotation test of the right shoulder 720 without weight. Alternatively, the subject may hold a weight in the right hand and perform the same motion as described above to perform the external rotation test of the right shoulder 720 with weight.

In another implementation, the subject may have a sensor attached to a left arm of a left shoulder 730 and perform the corresponding motion as described above to perform the external rotation test of the left shoulder 730 without weight. Alternatively, the subject may hold a weight in the left hand and perform the corresponding motion to perform the external rotation test of the left shoulder 730 with weight.

The acceleration of gravity in three axes relative to a known reference provides a signature of arm position during a forward extension test and/or an external rotation test. In the description below, the acceleration of gravity is used as an example. Similar description may exist for the angular velocity obtained from a gyroscope.

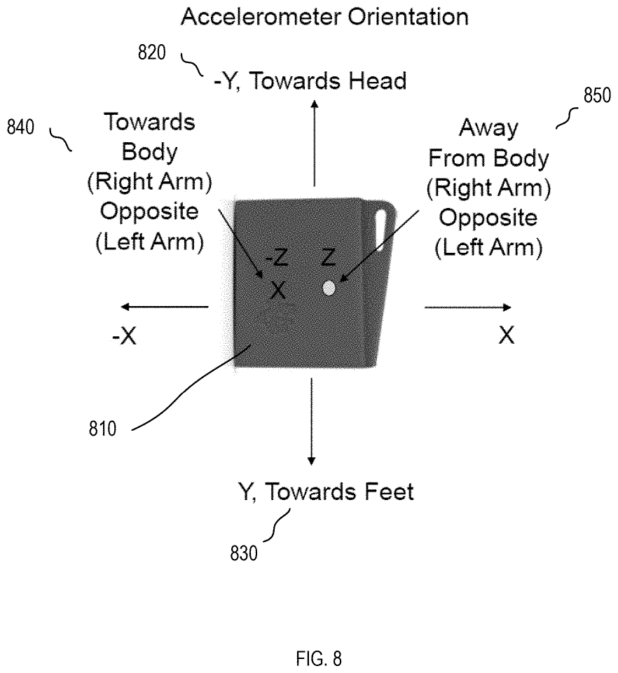

FIG. 8 shows an exemplary implementation of accelerometer's orientation. An accelerometer 810 may be disposed inside the sensor enclosure, and the sensor enclosure may be attached to a subject as shown in FIG. 5. The accelerometer may be fixed to the inside of the right arm of the subject between the bicep and triceps with the arm hanging at the subject's side. In this orientation, the accelerometer's −y direction may point towards the head of the subject. The accelerometer's y direction may point towards the feet of the subject. The accelerometer's z direction may point away from the body (when the sensor is attached to the right arm), and the accelerometer's −z direction may point towards the body (when the sensor is attached to the right arm).

When the sensor is attached to the left arm of the subject in the sulcus between the bicep and triceps with the arm hanging at the subject's side, the y and −y directions may remain the same, and the z and −z directions may change to its opposite direction.

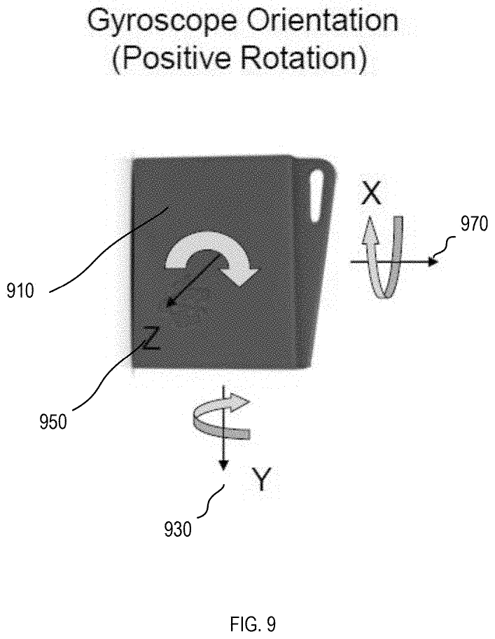

FIG. 9 shows an exemplary implementation of gyroscope's orientation. A gyroscope 910 may be disposed inside the sensor enclosure, and the sensor enclosure may be attached to a subject as shown in FIG. 5. The gyroscope may provide angular velocity of the sensor about each of three perpendicular axes. The gyroscope may provide an angular velocity about a first axis 930 (e.g. aligned with y direction), an angular velocity about a second axis 950 (e.g. aligned with the z direction), and an angular velocity about a third axis 970 (e.g. aligned with the x direction).

The system may be capable of synchronizing video obtained by at least one video camera with the kinematic data from the sensors. The video may provide visually captured movement in conjunction with the kinematic data. For example, a marker may be displayed on the kinematic data that corresponds the currently displayed frame of the video.

The video may provide a physician visualization of the movements in actual time or slow motion while reviewing the kinematic data, so that the video may aid the diagnostic analysis and train the physician to observe specific motions. These specific motions may be previously overlooked without the aid of video.

In one implementation, the synchronization of the video camera and the sensors may occur with a simple clap of hands. The audio of clapping hands from the video and the movement data of clapping hands from the sensors may be aligned/synchronized by with by hand adjustment or by computer programming. In another implementation, the synchronization of the video camera and the sensors may be achieved manually.

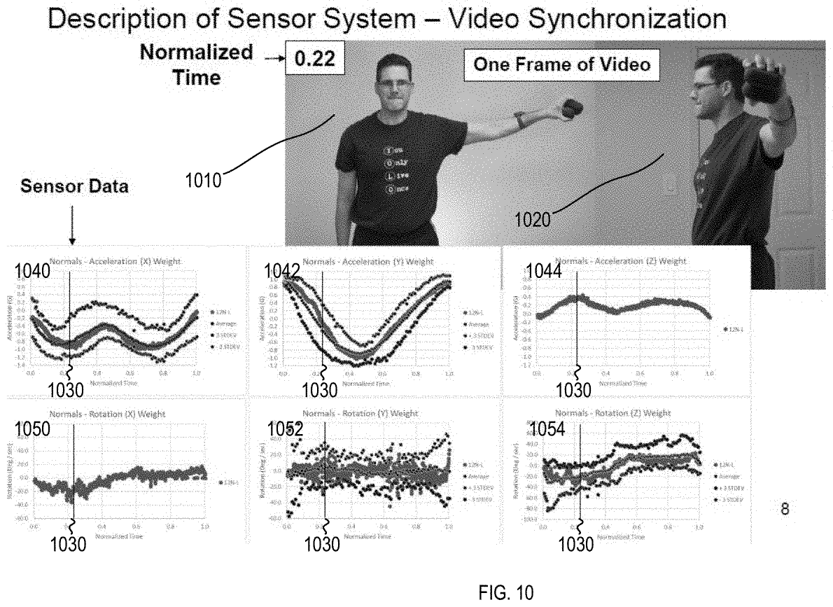

FIG. 10 shows an exemplary embodiment of synchronizing videos from video cameras with kinematic data from the sensors. Two video cameras may be used to record a front video from the front of the subject and a side video from the left side of the subject. A frame 1010 from the front video and a frame 1020 from the side video may be synchronized with a time point 1030 in acceleration data along x-axis 1040, acceleration data along y-axis 1042, acceleration data along z-axis 1044, rotation speed data along x-axis 1050, rotation speed data along y-axis 1052, and rotation speed data along z-axis 1054. In one implementation, the two videos may be synchronized to the kinematic data according to noise in the two videos and the kinematic data.

Optionally, the sensor may include a magnetometer. The magnetometer data obtained from the magnetometer may be analyzed together with the accelerometer and gyroscope data, which may enable the creation of an animated video to aid the physician in diagnosis of abnormal and normal conditions.

A group of known normal subjects may perform the tests to generate normal signatures. Another group of known abnormal subjects may perform the same tests to generate abnormal signatures. Normal signatures and/or abnormal signatures may be stored and labeled, and may be associated with groups and subgroups, for example and not limited to, normal groups and/or abnormal groups. The normal signatures and/or abnormal signatures may be also grouped and labeled based on their corresponding age ranges, gender, athletic sport or position, etc.

Several methods may be used to create a set of thresholds to distinguish the normal signatures from the abnormal signatures. The sets of thresholds may be stored and labeled based on their corresponding age ranges, gender, athletic sport or position, etc.

The first method may employ an average and a variance of the normal and/or abnormal signatures to create thresholds.

The second method may visually group normal and/or abnormal signatures to determine specific thresholds for normal and/or abnormal signatures. In one implementation, normal and abnormal signatures may be classified into two groups by the thresholds: one group is normal and the other group is abnormal.

The third method may employ advanced machine learning and statistical analysis to obtain thresholds so that they may distinguish between normal and abnormal characteristics. In one implementation, a database of doctors' previous observations may be very large and may be difficult or even impossible for a human being to access, and thus, machine learning based diagnosis may provide immediate access to the large database. In another implementation, some diagnostic studies may reveal that deep learning systems may correctly detect a disease state with a high accuracy, for example, as high as 87%—compared with 86% for healthcare professionals; and may correctly give all-clear results 93% of the time, compared with 91% for human experts.

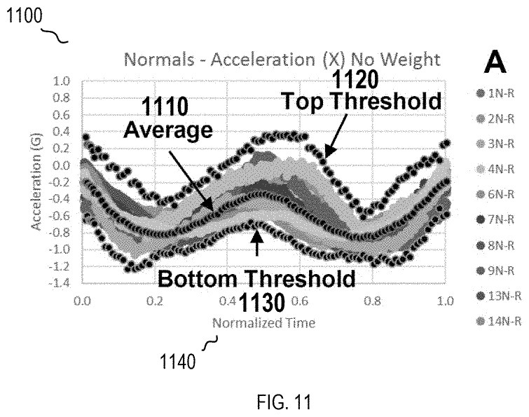

In one embodiment as shown in FIG. 11, a graph 1100 shows acceleration signatures along x-axis as a function of time for a number (N) of normal subjects during forward extension tests, for example, N=18. For each time point, an average value may be calculated based on all acceleration values of the N normal subjects, to obtain an average acceleration signature 1110 along x-axis as a function of time for the N normal subjects. Following the similar method, for each time point, a standard deviation value may be calculated based on all acceleration values of the N normal subjects, to obtain a standard derivation signature along x-axis as a function of time for the N normal subjects.

In this embodiment, the high/top threshold 1120 may be obtained based on the average acceleration signature and the standard derivation signature, for example but not limited to, the top threshold=average signature+3* standard derivation signature.

In this embodiment, the low/bottom threshold 1130 may be obtained based on the average acceleration signature and the standard derivation signature, for example but not limited to, the bottom threshold=average signature−3* standard derivation signature.

Optionally, the top threshold and bottom threshold may be further refined/adjusted to narrow threshold band as the database is increased. In one implementation, with larger sample sizes, for example but not limited to, 50, 100, 200, 400, etc., a different but more complex statistical approach may be used based on the larger sample sizes. The goal is to use a simple statistical approach to achieve an effective result. There is always a trade-off between high confidence and cost of obtaining data, particularly in clinical studies.

In one implementation, the normal or abnormal subjects may perform each test at their own pace within a time duration limit, and thus, the exact time duration of each test for each subject may not be the same. In this implementation, time normalization may be used to normalize time so that motion signatures from normal or abnormal subjects may be aggregated and/or compared to each other. In other words, comparison of normal and abnormal data, for example, magnitudes of acceleration and angular rotation, may be performed with a common time reference 1140 (e.g., normalized time). For example, time normalization may be performed by converting the duration of a test to a decimal between and including 0 and 1, which can be achieved mathematically by dividing each time point by the total time duration. Further, the data may be shifted, if needed, to synchronize the starting point of the tests prior to normalization.

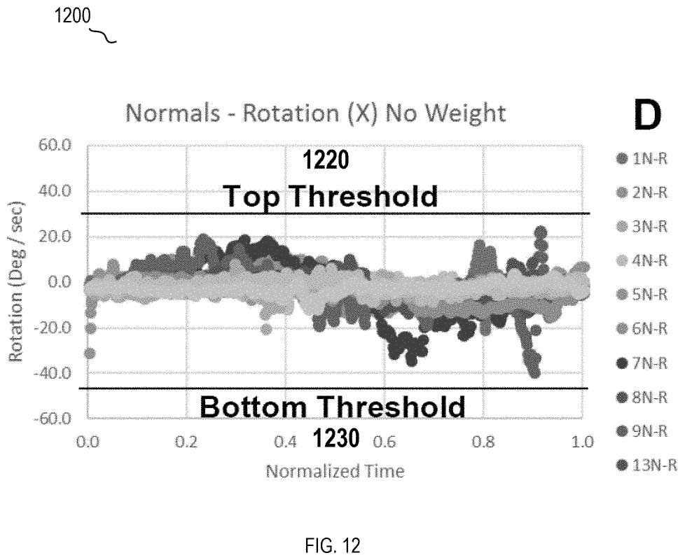

In one embodiment as shown in FIG. 12, a graph a graph 1200 shows rotational speed signatures along x-axis as a function of time for a number (N) of normal subjects during external rotation tests, for example, N=18. A grouping method may be used to visually obtain two lines on high and low edges of the rotational speed signatures. The high/top threshold 1220 is a line on the high edge with some space for additional variation. The low/bottom threshold 1230 is a line on the low edge with some space for additional variation.

The selection of the top threshold and bottom threshold may be refined as more motion signatures of normal subjects under the same tests are obtained.

The selection of the top threshold and bottom threshold may be tested and/or adjusted with motion signatures of abnormal subjects for the same tests.

The top and bottom threshold obtained in FIGS. 11 and 12 may be further refined with advanced statistical techniques and machine learning methods.

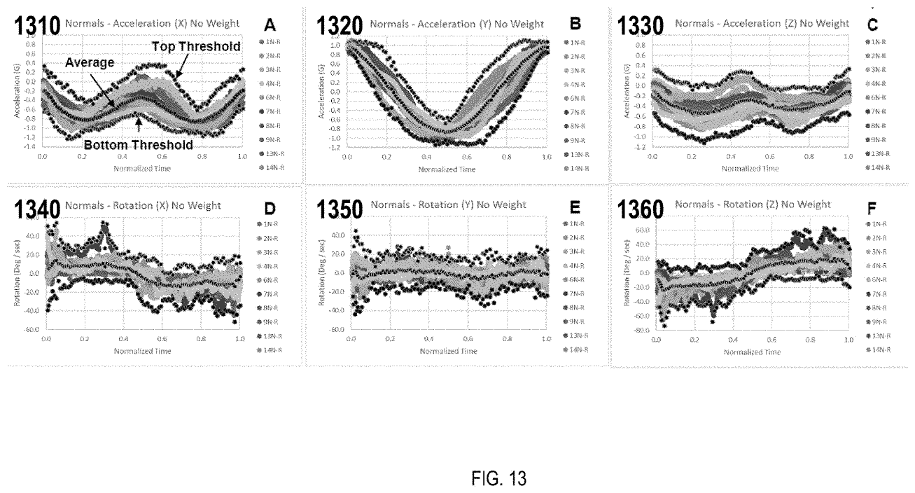

FIG. 13 is a set of graphs illustrating motion signature components. The motion signatures from 18 normal subjects may be obtained during forward extension tests without weight and their corresponding top and/or bottom thresholds. The motion signatures may include the acceleration signatures along one or more axes, for example, along x-axis 1310, along y-axis 1320, and along z-axis 1330. The motion signatures may also include the rotational speed signatures along one or more axes, for example, along x-axis 1340, along y-axis 1350, and along z-axis 1360.

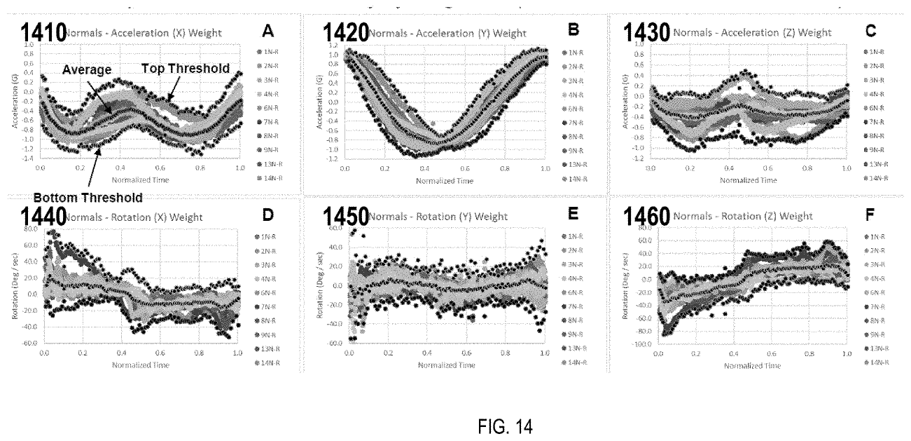

FIG. 14 is a set of graphs illustrating motion signature components. The motion signatures from 18 normal subjects may be obtained during forward extension tests with weight of about 5 lbs or more depending upon the strength of the subject held in their hand and their corresponding top and/or bottom thresholds compared to those without the weight. The motion signatures may include the acceleration signatures along one or more axes, for example, along x-axis 1410, along y-axis 1420, and along z-axis 1430. The motion signatures may also include the rotational speed signatures along one or more axes, for example, along x-axis 1440, along y-axis 1450, and along z-axis 1460. In another implementation, the weight used by the subject may vary. The weight may be greater in some subjects who are strong athletes.

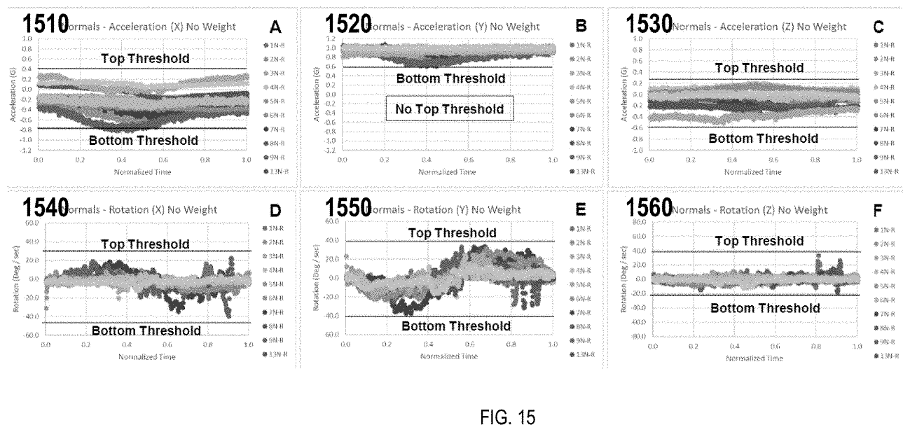

FIG. 15 is a set of graphs illustrating motion signature components. The motion signatures from 16 normal subjects may be obtained during external rotation tests without weight and their corresponding top and/or bottom thresholds. The motion signatures may include the acceleration signatures along one or more axes, for example, along x-axis 1510, along y-axis 1520, and along z-axis 1530. The motion signatures may also include the rotational speed signatures along one or more axes, for example, along x-axis 1540, along y-axis 1550, and along z-axis 1560.

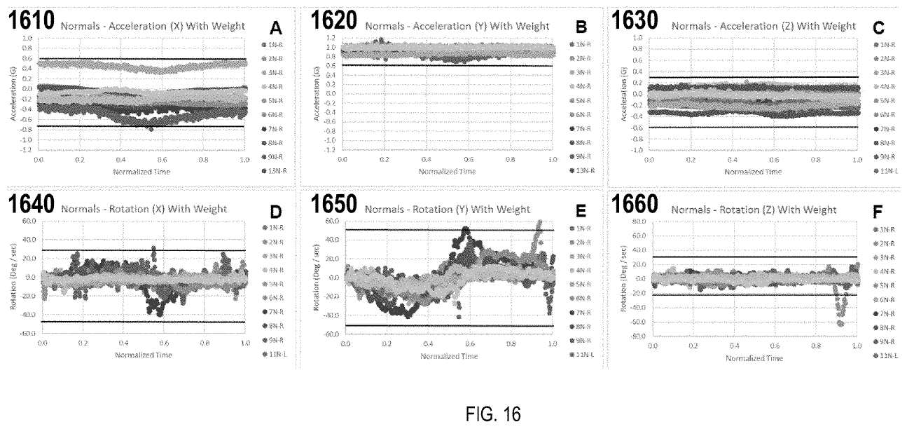

FIG. 16 is a set of graphs illustrating motion signature components. The motion signatures from 16 normal subjects may be obtained during external rotation tests with weight of about 5 lbs or more depending upon the strength of the subject held in their hand and their corresponding top and/or bottom thresholds compared to those without the weight and their corresponding top and/or bottom thresholds. The motion signatures may include the acceleration signatures along one or more axes, for example, along x-axis 1610, along y-axis 1620, and along z-axis 1630. The motion signatures may also include the rotational speed signatures along one or more axes, for example, along x-axis 1640, along y-axis 1650, and along z-axis 1660. In another implementation, the weight used by the subject may vary. The weight may be greater in some subjects who are strong athletes

FIGS. 13-16 show the motion signatures and their corresponding thresholds for different tests of the right shoulder. Alternatively, the motion signatures for tests of left shoulder may be analyzed and processed in a similar manner to obtain their corresponding top and/or bottom thresholds.

The top/bottom thresholds of each test in FIGS. 13-16 may be used to distinguish motion signatures of normal subjects from that of abnormal subjects. The top/bottom thresholds may be used to diagnose a subject based on motion signatures of the subject. In one implementation, a top/bottom thresholds as in FIGS. 13-16 may identify close to 100% of normal and abnormal motion signatures, for example, 98%. For the very few case (<2%), advance imaging and physician discernment may be used to make a determination. With more kinematic data from more normal and/or abnormal subjects, this percentage may increase to greater than 99% of cases. When the tears are small, a physician may not make a determination by mere physical examination, and ultrasound, MM, arthrogram, or arthroscopy may be required to confirm the diagnosis to make a determination.

The diagnosis result may be quantified by a diagnosis scale for musculoskeletal conditions of the suspected. The diagnosis scale may indicate a likelihood of injury requiring surgical intervention. The diagnosis scale may be obtained based on the magnitude and/or quantity of the deviation of the motion signatures of the subject relative to the corresponding top/bottom threshold.

The diagnosis scale may help a physician to determine whether an Mill is pursued or no further action other than physical therapy is needed.

In one implementation, the deviation of the motion signatures of the subject relative to the corresponding top/bottom threshold may be quantified as the amount of deviation from average route, similar to an area under a curve. The larger and longer the deviation from top/bottom thresholds from predetermined parameters that were considered average, the higher the likelihood of injury and a larger number for the diagnosis scale. Sophisticated algorithms may be used to obtain the diagnosis scale in this implementation.

In another implementation, the deviation of the motion signatures of the subject relative to the corresponding top/bottom threshold may be quantified by calculating variance. Higher variance of the deviation, the higher the likelihood of injury and a larger number for the diagnosis scale.

In another implementation, larger variance from normal in both acceleration and gyroscope data (e.g. corresponding to arm shakes) may be an indication of muscle injury. In one instance, this may be a sign of weakness especially in elderly, a sign of another underlying disease, or other possibilities. With other medical history and observation, a trained physician may determine whether it is weakness, underlying disease, or other cause that leads to the larger variance. In another instance, the larger variance may help in diagnosis when the larger variance is seen with other signs of injuries, for example but not limited to, crossing thresholds, signature not following trend line and barely within thresholds.

In the system for dynamic diagnosis, more than just a few points beyond the top/bottom thresholds may be needed to classify motion signature of a suspect subject as an abnormal signature. In one embodiment, in order to determine an abnormal signature, more than one (e.g., typical values including two and three) graph having many points beyond the top/bottom thresholds and other points are not following normal signatures may be needed for dynamic diagnosis of abnormal musculoskeletal conditions.

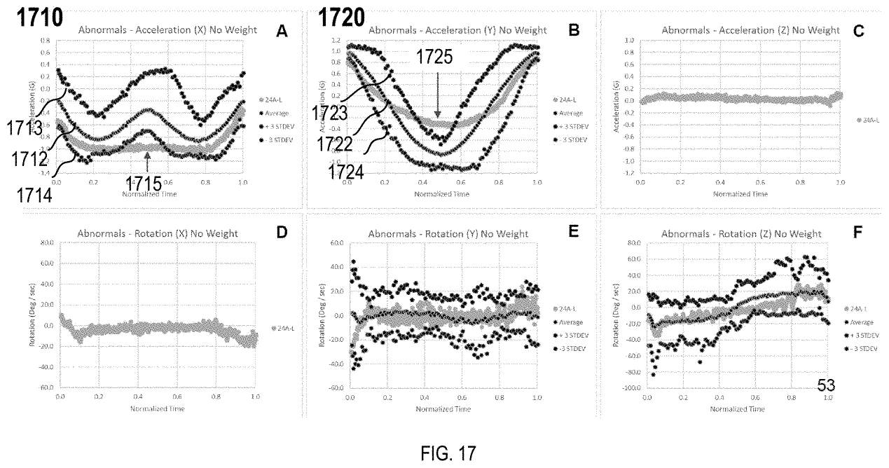

FIG. 17 is a set of graphs illustrating motion signature components. The motion signatures from an abnormal subject may be obtained during forward extension tests without weight. MRI diagnosis for the abnormal subject may include full thickness tear of a supraspinatus injury, tendinosis of subscapularis, AC joint osteoarthritis.

A top threshold and/or bottom threshold may be obtained based on motion signatures from normal subjects during forward extension tests without weight. For an example in FIG. 17, the acceleration signature along x-axis 1710 includes a top threshold 1713, an average 1712, and a bottom threshold 1714. For the acceleration signature along x-axis 1710, the deviation may include at least one area: multiple points 1715 may be beyond the bottom threshold 1714, e.g., below the bottom threshold 1714. The acceleration signature along y-axis 1720 in FIG. 17 includes a top threshold 1723, an average 1722, and a bottom threshold 1724. For the acceleration signature along y-axis 1720, the deviation may include at least one area: multiple points 1725 may be beyond the top threshold 1723, e.g., above the top threshold 1723. Based at least on the above deviation, the system may dynamically diagnose as potential for injury, not following normal trends and outside thresholds.

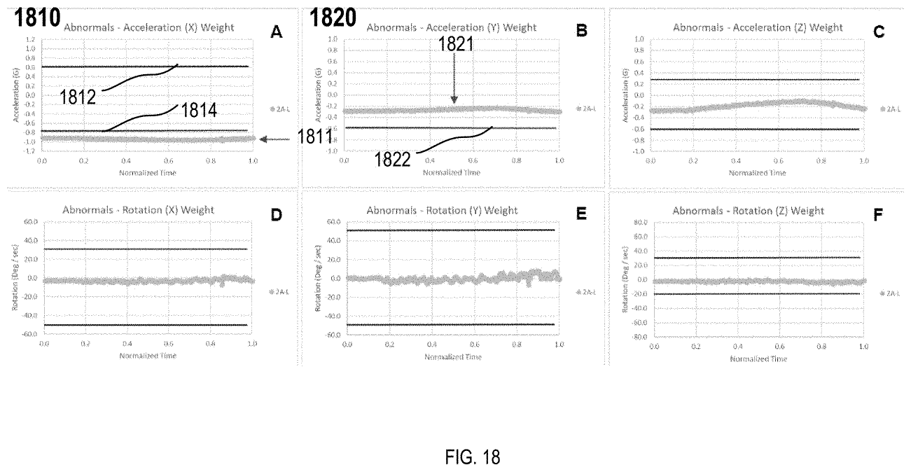

FIG. 18 is a set of graphs illustrating motion signature components. The motion signatures from an abnormal subject may be obtained during external rotation tests with a weight. MM diagnosis for the abnormal subject may include high grade partial thickness tear of a supraspinatus tendon, and/or reactive bursitis.

A top and/or bottom thresholds may be obtained based on motion signatures from normal subjects during external rotation tests with a weight. For example, the acceleration signature along x-axis 1810 includes a top threshold 1812 and a bottom threshold 1814. For the acceleration signature along x-axis 1810, a deviation may include almost all points 1811 being beyond (below) the bottom threshold 1814. The acceleration signature along y-axis 1820 includes a top threshold 1822. For the acceleration signature along y-axis 1820, a deviation may include almost all points 1821 may be beyond (above) the top threshold 1822. Based at least on the above deviations, the system may dynamically diagnose the subject as abnormal conditions with or without injury.

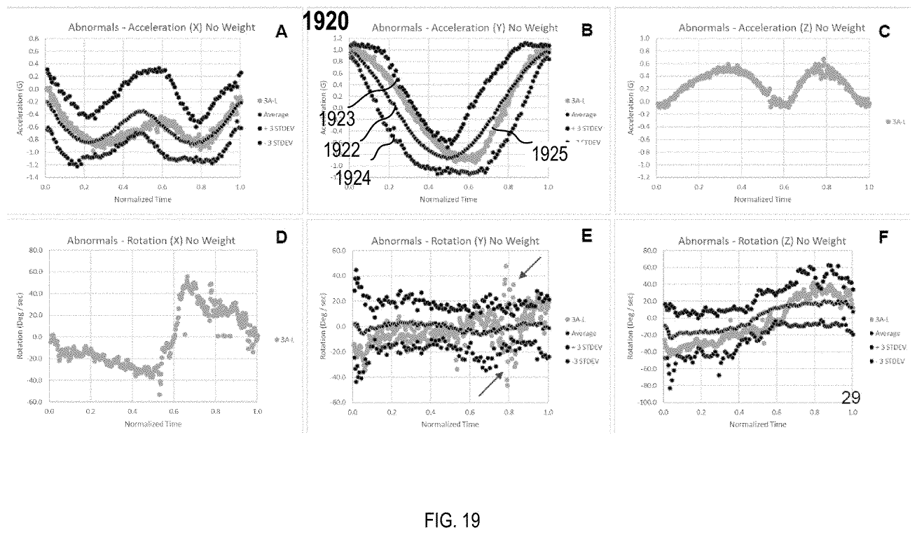

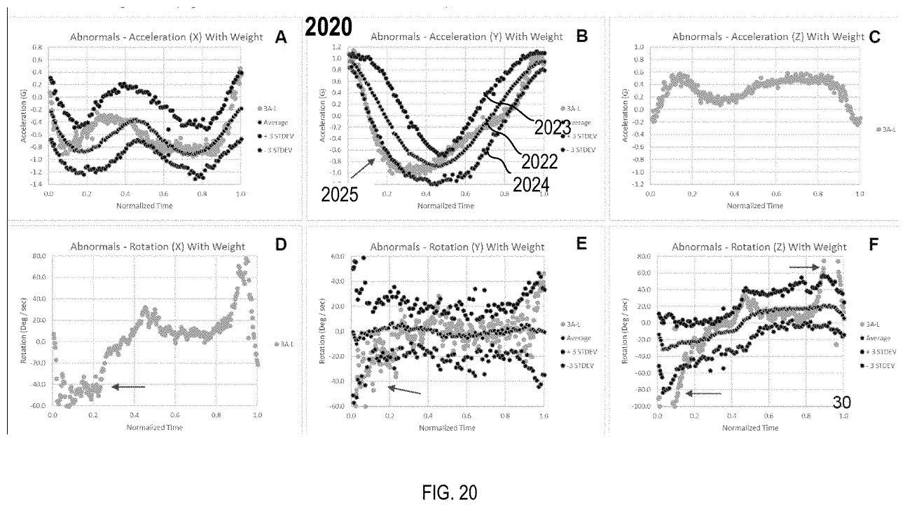

The presence of weight may make a difference in the motion signatures during a test. For example, FIGS. 19-20 show that the presence of weight makes a difference in the motion signatures for an abnormal subject during the forward extension test.

FIG. 19 is a set of graphs illustrating motion signatures obtained from an abnormal subject during forward extension tests without weight. FIG. 20 is a set of graphs illustrating motion signatures obtained from the abnormal subject during forward extension tests with weight. The motion signatures in FIG. 19 are different from the motion signatures in FIG. 20.

For example but not limited to, the acceleration signature along y-axis 1920 in FIG. 19 is within a top threshold 1923 and a bottom threshold 1924, where the top threshold 1923 and the bottom threshold 1924 have an average 1922. The acceleration signature along y-axis 2020 in FIG. 20 is quite different from the acceleration signature along y-axis 1920 in FIG. 19. Multiple points of multiple points 2025 of the acceleration signature along y-axis 2020 may be beyond the bottom threshold 2024, e.g., below the bottom threshold 2024, having a top threshold 2023 and an average 2022.

### Embodiment for Dynamic Diagnosis of Musculoskeletal Conditions of Elbow

The present disclosure also describes an embodiment for dynamic diagnosis of elbow conditions. This embodiment may include features similar to the above embodiment for dynamic diagnosis of shoulder conditions, and those similar features are not repeated again.

This embodiment may include many modules and many tests, not just one test as with the torso in activities of daily living. The embodiment may include an activity of daily living (ADL) limitation related to a specific diagnosis.

In one implementation, the motion pattern of a first test for elbow motion and secondarily for the biceps muscle may include, with or without resistance of a weight (5-10 lbs) in a hand of a subject, sitting or standing with spine erect, extending an elbow with the upper extremity at their side with the hand adjacent to the lateral hip, facing the palm forward so the forearm is supinated, keeping elbow at side, raising the hand upward as far as possible and back down without moving position of the elbow, and thereby testing elbow flexion.

During the first test, one sensor may be attached to the wrist of the subject. Optionally, another sensor may be attached on the arm to assess the arm motion to assist in elbow flexion.

In another implementation, the motion pattern of a second test may be specifically for the ulnar collateral ligament of the elbow and often torn in throwing sports. The motion pattern of the second test may include standing, starting a position with an arm at 90 degrees elevated and an elbow bending at 90 degrees, performing external rotation of the arm to maximum, then performing internal rotation to a maximum degree, and returning to the starting position.

During the second test, one sensor may be attached on the dorsum of the wrist by which rotation of the forearm would be recorded.

In another implementation, the motion pattern of a third test may be specifically for throwing motion. The motion pattern of the third test may include, standing, starting with replicating a throwing motion, and throwing motion with turn-on at point of maximal external rotation.

During the third test, one sensor may be attached on a back of the wrist.

The musculoskeletal conditions may include osteochondritis dissecans, arthritis, radial head subluxation, and loose body related to elbow joint, biceps muscle/tendon, triceps muscle/tendon, and ulnar collateral ligament. For the evaluation of technique or compensatory motion, a sensor may be placed on the head or over the spine.

The deviation of the motion signature of a subject from the normal signatures may be quantified in various percentages or distances to reflect the different locations of deficient and magnitude. An initial deviation may be quantified on any three or more of groups of motion signatures.

The kinematic data and the corresponding motion signatures may be recorded by the Apple watch and viewed on the cell phone or computer screen. The method may include determining the significant deviations in each of the 4 parameters: acceleration, rotation, deviation of plane, and timing. A four-dimensional stick figure video (includes time in space) of the motion may be constructed in color verse the composite of normal in black.

The normal signatures and the corresponding top/bottom thresholds may be updated constantly based on an ever-expanding database.

### Embodiment for Dynamic Diagnosis of Musculoskeletal Conditions of Knee Joint and Adjacent Structures

The present disclosure also describes an embodiment for dynamic diagnosis of musculoskeletal conditions of knee joint and adjacent structures. This embodiment may include features similar to the above embodiments for dynamic diagnosis of musculoskeletal conditions, and those similar features are not repeated again.

This embodiment may include many modules and many tests, not just one test as with the torso in activities of daily living. The embodiment may include an activity of daily living (ADL) limitation related to a specific diagnosis.

A number of tests may be performed with and without resistance of a weight (5-10 lbs) attached to a boot of a subject.

The motion pattern of a first test may be specifically for knee joint dynamics through the range of motion and dynamic motion and secondarily for the quadriceps muscle. The motion pattern of the first test may include: sitting with a knee at the break of the bench or chair and the leg hanging in passive flexion, starting in a position with the knee bent with the leg hanging in a passive flexion, extending the knee by lifting the foot into extension, and returning to the starting position. Another would be with the patient prone and flexing the knee or any other test position common to the physical examination.

During the first test, one sensor may be attached on the distal leg and/or the tibial bone just above the ankle.

The motion pattern of a second test may be specific for the hamstring muscles/tendon. The motion pattern of the second test may include, positioning the subject being prone on an exam table, starting in a position with a lower extremity in extension, performing a knee flexion to a maximal active position, and returning to the starting position.

During the second test, one sensor may be attached on the distal leg and/or the tibia just above the ankle.

The motion pattern of a third test may be specific for the anterior cruciate ligament insufficiency. The motion pattern of the third test may include, sitting with a knee at the break of the bench or a chair and the leg hanging in passive flexion, starting with a knee at 90 degrees flexion, and pushing against stationary resistance to extension.

During the third test, one sensor may be attached on the tibia just below the knee. The third test may be used to perform a specific diagnosis of anterior cruciate tear.

The motion pattern of a fourth test may be specific for the posterior cruciate ligament insufficiency. The motion pattern of the fourth test may include, sitting with a knee at the break of the bench or chair and the leg hanging in passive flexion, starting with a knee at 90 degrees flexion, and pushing against stationary resistance to flexion.

During the fourth test, one sensor may be attached on the tibia just below the knee. The fourth test may be used to perform a specific diagnosis of posterior cruciate tear. To test for the posterior cruciate deficiency the reverse of the above testing for the anterior cruciate ligament would be performed.

The motion pattern of a fifth test may be specific for the patellar instability. The sensors would be in similar position. The motion pattern of the fifth test may include, sitting with a knee at the break of the bench or chair and the leg hanging in passive flexion, starting with a knee at 90 degrees flexion, extending the knee and returning to starting position, and/or pushing against stationary resistance with knee at 30 degrees of flexion.

During the fifth test, one sensor may be attached on the tibia just below the knee. The fifth test may be used to perform a specific diagnosis of patellar instability.

The musculoskeletal conditions of knee may include normal joint without arthritis, patellar subluxation, patellar dislocation history, patellar position baja or alta, torn ligaments of each kind and then combinations, varus, valgus deformity, etc.

Embodiment for Dynamic Diagnosis of Musculoskeletal Conditions of Achilles Tendon and/or Gastrocnemius

The present disclosure also describes an embodiment for dynamic diagnosis of musculoskeletal conditions of Achilles tendon and/or gastrocnemius. This embodiment may include features similar to the above embodiment for dynamic diagnosis of musculoskeletal conditions, and those similar features are not repeated again.

This embodiment may include many modules and many tests, not just one test as with the torso in activities of daily living. The embodiment may include an activity of daily living (ADL) limitation related to a specific diagnosis.

A number of tests may be performed with and without resistance of a weight (5-10 pounds) attached to a boot of a subject.

The motion pattern of a first test may be specifically for musculotendinous function and secondarily for ankle joint dynamics through the range of motion; e.g., of motion and dyna motion and secondarily for the quadriceps muscle. The motion pattern of the first test may include: standing, start standing on both feet, performing to go up on their tip toes and return to standing. The second phase of this function would be to stand on the floor with both feet and elevate the forefoot off the floor. This would be repeated with each limb individually to assess difference from the control opposite limb.

During the first test, one sensor may be attached on the tibial bone just above the ankle.

The motion pattern of a second test may be specifically specific for each Achilles/gastrocnemius muscles/tendon. The motion pattern of the second test may include: standing, start with a lower extremity in extension, performing to bend opposite knee and then to go up on toes with foot that remains on the floor, and returning to the starting position. The second phase of this function would be for them to stand on the floor with one leg.

During the second test, one sensor may be attached on the tibial bone just above the ankle.

The first and/or second may be repeated for the opposite leg for control or pathology.

The musculoskeletal conditions may include normal joint without arthritis, Achilles tendon tear, gastrocnemius tear, etc.

### Embodiment for Dynamic Diagnosis of Musculoskeletal Conditions of Ulnar Collateral Ligament

The present disclosure also describes an embodiment for dynamic diagnosis of musculoskeletal conditions of ulnar collateral ligament (UCL). This embodiment may include features similar to the above embodiment for dynamic diagnosis of musculoskeletal conditions, and those similar features are not repeated again.

The injury to the ulnar collateral ligament (UCL) of the elbow may be a serious problem in professional athletes such as baseball pitchers, and may be rare in other types of sport throwing and among those playing other positions in baseball. A biomechanical load on UCL on the inside of the elbow may be greatest at the moment when the pitcher takes his/her arm forward from the cocking position. This destructive force may be described as valgus extension overload syndrome. The mean valgus stress per pitch in an adult may be about 64 N·m, which may exceed the restraining force of UCL as an ultimate valgus torque of the UCLmay be about 33 N·m. Consequently, a 64 N·m varus counter torque may be needed to resist this massive torque. For example, a 64 N·m varus torque applied to the elbow may be equivalent to holding the weight of 150 baseballs. The UCL alone provides 54% of this varus counter torque or roughly 34 N·m. One reason that this ligament is not more commonly torn may be that UCL complex may be protected by arm muscle activity termed dynamic stabilization, provided by the triceps, anconeus, flexor-pronator mass, and internal rotation of the shoulder.

In addition, there may be individual variation in the physical properties of ligamentous tissue from one human to another. The most obvious clinical manifestation of loose ligamentous tissue may be related to genetically weak or lax ligaments, for example, Ehler Danlos Syndrome or the Marfan Syndrome. The individual variations and potential vulnerability of the UCL tissue on those in baseball pitching may be assessed for those with risk of the injury. The condition, although publicized in professional players, may not be restricted to the professional players; and there may be rising incidence of young men baseball pitchers. Although there may be a successful reconstruction operation for the professional pitcher, a hand surgeon may remain a problem of prediction of vulnerability and prevention, especially in the vulnerable youth. The diagnosis of UCL may not be easily made except in rare conditions of complete tear. Comparison stress tests measured by radiology may not be clinically sensitive, not be easily documented, and may have variations from one examiner to another. A laxity of 0.5 millimeter may be considered as very small to be detected and may exist as a normal variation.

There may remain the difficulty of UCL diagnosis by clinical assessment and imaging techniques. There may be a need for a more sensitive means of assessing the status and perhaps more importantly the vulnerability of young baseball pitchers. A prophylactic assessment may also be needed. Non-surgical treatment methods of physical therapy and modification of throwing motion may exist, and there may be no existing means of measuring the progress or safety of time to return to pitching. The present disclosure describes an embodiment providing a more sensitive data collection and reporting instrument to complement the clinical impression in decision making, one without examiner prejudice or patient's malingering.

The Micro-Electro-Mechanical System (MEMS) sensor technology used in the present disclosure may provide measurements of various types of measurements, including accelerometer/acceleration, gyroscope/rotational speed, magnetometer/earth magnetic field. The MEMS technology may provide a means of testing the integrity of the musculoskeletal system, including the UCL of the elbow. The embodiment may include a highly sensitive, specific, small/lightweight and reproducible method adapted for testing this ligament. The output of the sensors may provide a graphic entitled as a motion signature. This signature may be unique for each individual as a personal autograph. In one implementation, the signature may be used to determine normal UCL condition from abnormal condition. In another implementation, data from these sensors may be used to generate an animation of motion (visualization) that may be used in diagnostics by the physician. In another implementation, Bluetooth low energy wireless communication for the sensor system may be used to remove any motion restriction due to a connecting wire.

The present disclosure describe a simple, reproducible, sensitive data collection method to identify the status of the ulnar collateral ligament absent elaborate video testing or radiological imaging. In one implementation, the method may provide a simple, easy means to execute by non-professional staff in a short period of time. In another implementation, the method may be transportable to and/or used in any environment, for example, office, clinic, or playing field.

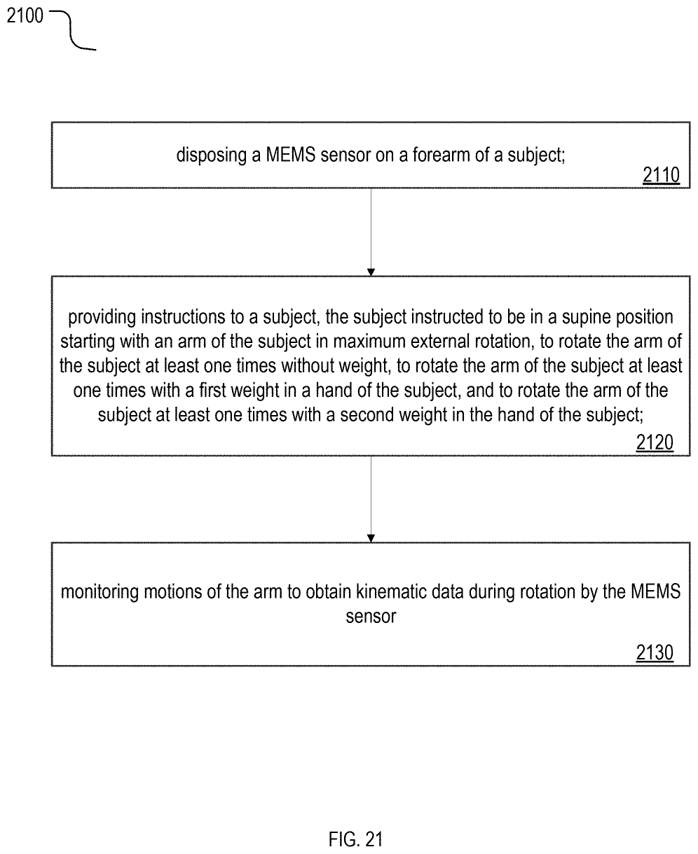

Referring to FIG. 21, a method 2100 for diagnosing musculoskeletal conditions of UCL may include step 2110: disposing a MEMS sensor on a forearm of a subject; step 2120: providing instructions to a subject, the subject instructed to be in a supine position starting with an arm of the subject in maximum external rotation, to rotate the arm of the subject at least one times without weight, to rotate the arm of the subject at least one times with a first weight in a hand of the subject, and to rotate the arm of the subject at least one times with a second weight in the hand of the subject; and step 2130: monitoring motions of the arm to obtain kinematic data during rotation by the MEMS sensor. In one implementation, the method 2100 may include one or more steps of any embodiment or a combination of embodiments as previously described.

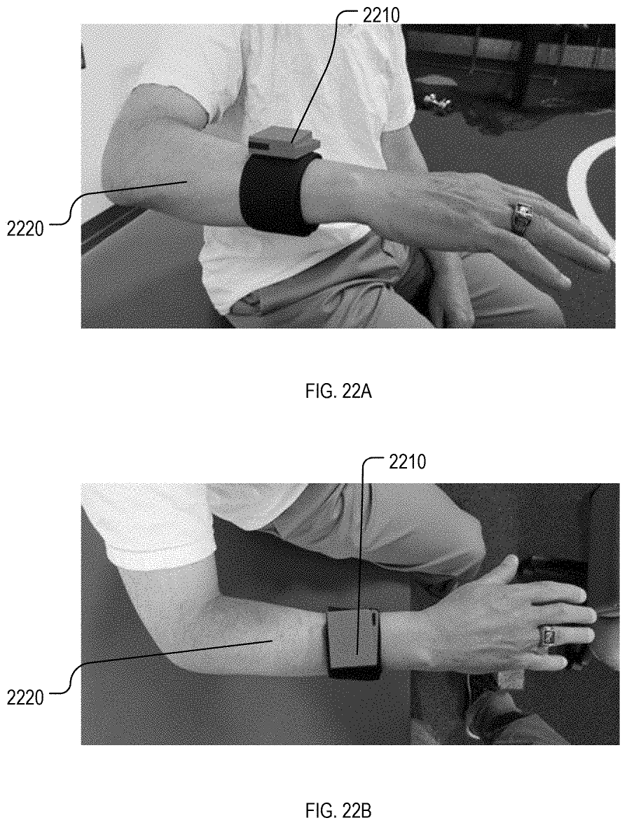

In step 2110, the MEMS sensor 2210 may be disposed on the forearm 2220 of the subject as shown in FIGS. 22A and 22B. The MEMS sensor 2210 may include an accelerometer and a gyroscope. In one implementation, the orientation of the MEMS sensor 2210 may be shown in FIGS. 8 and 9.

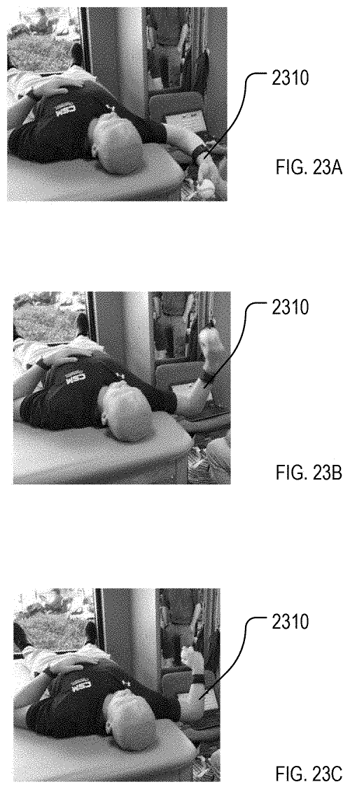

In step 2120, referring to FIG. 23A, the subject may be in a supine position as a starting position and the arm 2310 in maximum external rotation position. Referring to FIGS. 23B and 23C, the subject may be instructed to rotate the arm 2310 internally or externally. In one implementation, the subject may be instructed to rotate the arm three times at a slow comfortable pace. For example, the subject may rotate the arm 90 degrees and in the horizontal plane of the body (for example, table top). During the rotation, the subject may be instructed to hold the arm first at the 90-degree position, which may be the most stable position for the UCL. In another implementation, the subject may be instructed to rotate 70 and less degrees of flexion. In another implementation, the forearm of the subject may be held in neutral, maximal pronation, and then supination, which may be the least restraining on the UCL.

In step 2120, the subject may be instructed to rotate the arm under various conditions, for example, without weight, with a first weight, or with a second weight. In one implementation, the subject may be instructed to perform the test with no weight in the hand, with holding a baseball when the first weight is the baseball, and with grasping a 5-pound weight when the second weight is the 5-pound weight.

The rotation without any weight may be used as the control.

The grasping of the baseball may be the rotation with the first weight, as though to throw a fast ball and be used to contract upper extremity muscles that may counter instability. The weight of the baseball may be about 5 ounces. This minimal weight may not be likely alone to stress the UCL.

The 5-pound weight may be the rotation with the second weight to load the UCL beyond that of the first weight. In another implementation, variations in the weight, positions of the extremity and pace or range of the motions may be altered. Here in the present disclosure, “about” a value may be referred to a range between 90% and 110% of the value, inclusive.

In one implementation, this motion may be performed by the subject in relatively slow pace, for example, over a duration of from about 3 second to about 30 seconds (or greater), and the MEMS sensor may monitor the motion of the arm.

In step 2130, the acceleration of gravity may be measured in a plurality of axes (for example, x-axis, y-axis, and z-axis) by the accelerometers in the MEMS sensor. The rotation of the arm may be measured in a plurality of axes by the gyroscopes in the MEMS sensor.

The data obtained by the MEMS sensor may provide insight into the position of the forearm and any motion restrictions that may be present in an abnormal condition (for example UCL injury) in comparison to a normal non-injured person. For example, acceleration and/or rotational speed and direction can be affected by abnormal conditions in comparison to a normal non-injured person. In addition, accelerometers and gyroscopes may detect sudden changes in motion due to pain, laxity, stiffness, instability, stress/inability to support weight, etc.

The force that strains the UCL may be that of the forces created by the rapid deceleration/acceleration motion at the moment of change of direction from maximal cocking of the elbow to forward motion. Therefore, in one implementation, the test method may include a rotational movement of the upper arm so the forearm is moved from vertical backward to maximal external rotation position, followed by a smooth transition to a forward internal rotation moment of the arm (humerus). In another implementation, the test may be performed with each of the three weighted tests; nothing in hand, grasping baseball as for a fast ball pitch and then with a five-pound weight to load the ligament at transition. Each weighed test may be repeated a plurality of times (for example, three times).

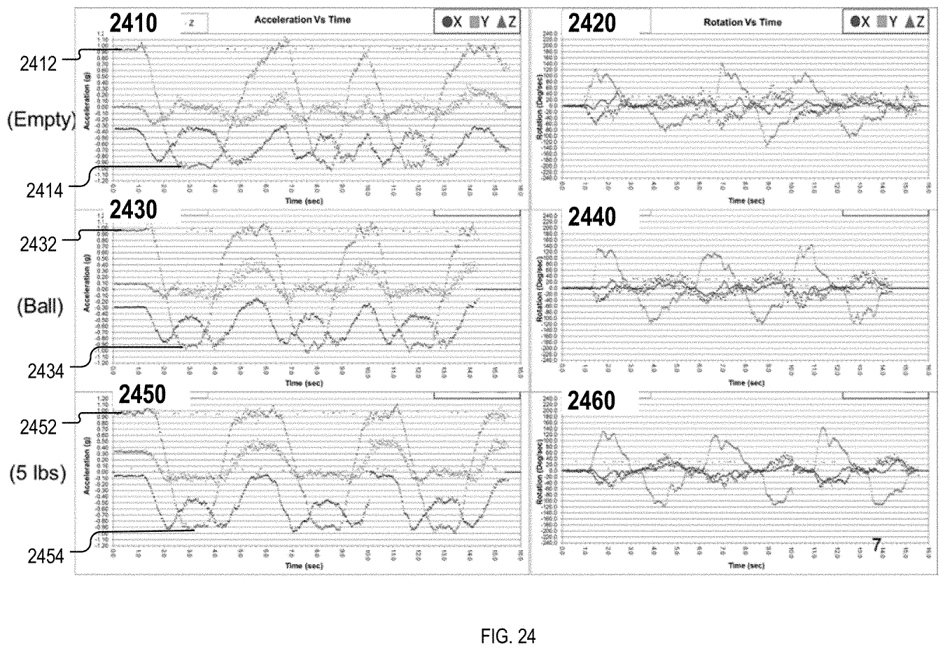

Referring to FIG. 24, in one implementation, charts of measured data by the MEMS sensor for one normal subject are shown. The charts may include acceleration data measured by an accelerator and rotation data measured by a gyroscope. Chart 2410 is the acceleration as a function of time when the subject performs the test without holding any weight. Chart 2420 is the rotation as a function of time when the subject performs the test without holding any weight. Chart 2430 is the acceleration as a function of time when the subject performs the test with holding a first weight (e.g., a ball). The ball may be a baseball with a weight of about 5 ounces. Chart 2440 is the rotation as a function of time when the subject performs the test with holding a first weight (e.g., a ball). Chart 2450 is the acceleration as a function of time when the subject performs the test with holding a second weight (e.g., a five-pound weight). Chart 2460 is the rotation as a function of time when the subject performs the test with holding a second weight (e.g., a five-pound weight). For each chart, data of three-dimensions may be shown, for example, x-axis data, y-axis data, and z-axis data.

The charts may be used to obtain the signature for the subject. For example but not limited to, the acceleration data along z-axis may be used for analysis. For chart 2410, the acceleration along z-axis may have a high value 2412 of about 1.0 g, and may have a low value 2414 of about −1.0 g. Thus, a range of motion of z-axis may be from −1.0 g to 1.0 g for a normal subject without holding any weight. Here, “g” may refer to the standard gravity with a value of 9.8 meters per second squared. Here “about” a value may be a range between 90% and 110% of the value, inclusive.

For chart 2430, the acceleration along z-axis may have a high value 2432 of about 1.0 g, and may have a low value 2434 of about −1.0 g. Thus, a range of motion of z-axis may be from −1.0 g to 1.0 g for a normal subject with holding a first weight (e.g., a ball).

For chart 2450, the acceleration along z-axis may have a high value 2452 of about 1.0 g, and may have a low value 2454 of about −1.0 g. Thus, a range of motion of z-axis may be from −1.0 g to 1.0 g for a normal subject with holding a second weight (e.g., a five-pound weight).

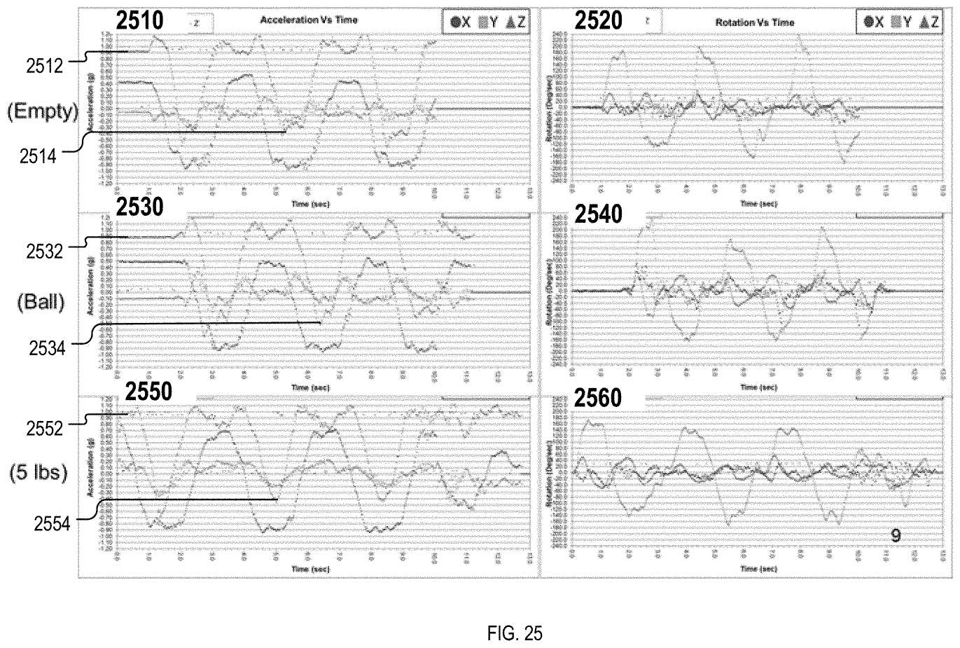

Referring to FIG. 25, in another implementation, charts of measured data by the MEMS sensor for one subject having UCL tears are shown. The charts may include acceleration data measured by an accelerator and rotation data measured by a gyroscope. Chart 2510 is the acceleration as a function of time when the subject performs the test without holding any weight. Chart 2520 is the rotation as a function of time when the subject performs the test without holding any weight. Chart 2530 is the acceleration as a function of time when the subject performs the test with holding a first weight (e.g., a ball). Chart 2540 is the rotation as a function of time when the subject performs the test with holding a first weight (e.g., a ball). Chart 2550 is the acceleration as a function of time when the subject performs the test with holding a second weight (e.g., a five-pound weight). Chart 2560 is the rotation as a function of time when the subject performs the test with holding a second weight (e.g., a five-pound weight). For each chart, data of three-dimensions may be shown, for example, x-axis data, y-axis data, and z-axis data.

The charts may be used to obtain the signature for the subject so as to diagnose musculoskeletal conditions of UCL of the subject. For example but not limited to, the acceleration data along z-axis may be used for analysis. For chart 2510, the acceleration along z-axis may have a high value 2512 of about 0.9 g, and may have a low value 2514 of about −0.4 g. Thus, a range of motion of z-axis may be from −0.4 g to 0.9 g for a subject having UCL tears without holding any weight.

For chart 2530, the acceleration along z-axis may have a high value 2532 of about 0.9 g, and may have a low value 2534 of about −0.5 g. Thus, a range of motion of z-axis may be from −0.5 g to 0.9 g for a subject having UCL tears with holding a first weight (e.g., a ball).

For chart 2550, the acceleration along z-axis may have a high value 2552 of about 1.0 g, and may have a low value 2554 of about −0.4 g. Thus, a range of motion of z-axis may be from −0.4 g to 1.0 g for a subject having UCL tears with holding a second weight (e.g., a five-pound weight).

Referring to FIGS. 24 and 25, there may be a clear restriction in motion of z-axis for the accelerations. For example, the acceleration along z-axis may be limited to about 70% of the normal range. The normal range may be from about −1.0 g to about 1.0 g.

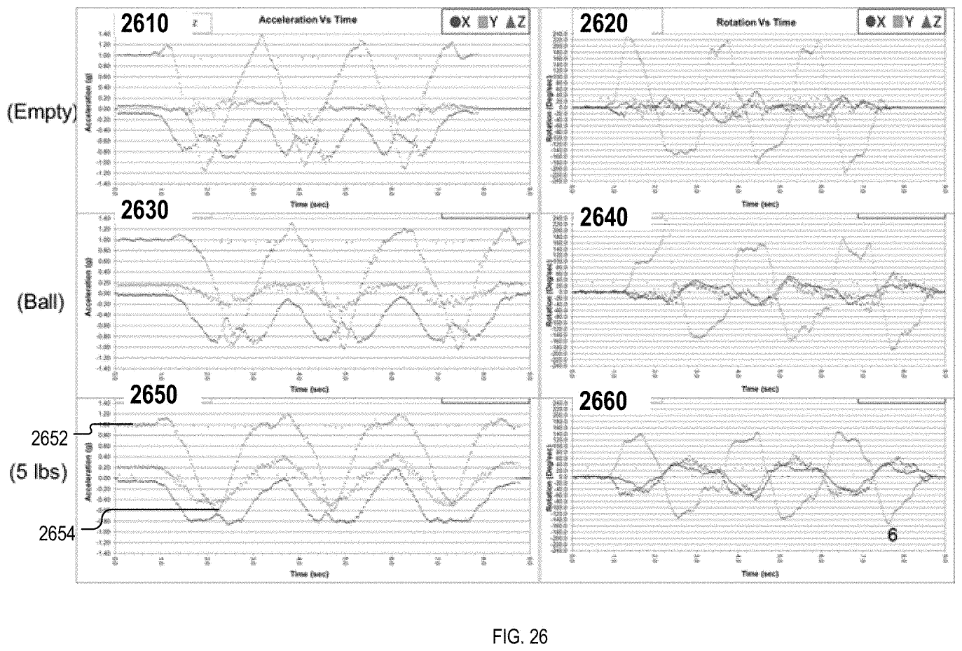

Referring to FIG. 26, in another implementation, charts of measured data by the MEMS sensor for another subject having UCL tears are shown. The charts may include acceleration data measured by an accelerator and rotation data measured by a gyroscope. Chart 2610 is the acceleration as a function of time when the subject performs the test without holding any weight. Chart 2620 is the rotation as a function of time when the subject performs the test without holding any weight. Chart 2630 is the acceleration as a function of time when the subject performs the test with holding a first weight (e.g., a ball). Chart 2640 is the rotation as a function of time when the subject performs the test with holding a first weight (e.g., a ball). Chart 2650 is the acceleration as a function of time when the subject performs the test with holding a second weight (e.g., a five-pound weight). Chart 2660 is the rotation as a function of time when the subject performs the test with holding a second weight (e.g., a five-pound weight). For each chart, data of three-dimensions may be shown, for example, x-axis data, y-axis data, and z-axis data.

The charts may be used to obtain the signature for the subject to diagnose musculoskeletal conditions of UCL of the subject. For example but not limited to, the acceleration data along z-axis may be used for analysis. In chart 2650, the subject may be under greater stress condition due to the five-pound weight. In chart 2650, the acceleration along z-axis may have a high value 2652 of about 1.0 g, and may have a low value 2654 of about −0.5 g. Thus, a range of motion of z-axis may be from −0.5 g to 1.0 g for a subject having UCL tears with holding the five-pound weight. Referring to FIGS. 24 and 26, there may be a clear restriction in motion of z-axis for the accelerations under a greater stress. For example, the acceleration along z-axis may be limited to about 80% of the normal range. The normal range may be from about −1.0 g to about 1.0 g. In one implementation, as shown in chart 2610 when weight is not used, the restriction in motion of z-axis may not be easily detected. In another implementation, a stress condition from without any weight to with a five-pound weight may be performed to determine abnormal conditions of UCL.

In another embodiment, a signature difference may be used to determine the musculoskeletal conditions of UCL and be used to determine abnormal from normal conditions. The detectability of these differences may be enhanced with other methodologies, for example but not limited to, statistics, machine learning, advanced computational analysis, or animation. Thresholds may be defined using these techniques to provide a diagnosis for the physician that is extremely easy to understand with minimal training. In addition, the coupling of MEMS technology with the clinical conditions may better assist in prevention, non-operative treatment, surgical indications and rehabilitation. In addition, return safely to play measurements may be identified. No other existing evaluation may provide the sensitivity, specificity and reliability as achieved by the present disclosure using MEMS sensor. This level of discernment may not be possible by physical examination, standard radiology or MM. Paramount in relevance and importance of this novel method of determining UCL injury may be the prevention of such injuries or the care thereof in the ever-expanding incidence among the youth.

While the particular disclosure has been described with reference to illustrative embodiments, this description is not meant to be limiting. Various modifications of the illustrative embodiments and additional embodiments of the disclosure will be apparent to one of ordinary skill in the art from this description. Those skilled in the art will readily recognize that these and various other modifications can be made to the exemplary embodiments, illustrated and described herein, without departing from the spirit and scope of the present disclosure. It is therefore contemplated that the appended claims will cover any such modifications and alternate embodiments. Certain proportions within the illustrations may be exaggerated, while other proportions may be minimized. Accordingly, the disclosure and the figures are to be regarded as illustrative rather than restrictive.

1. A method for dynamically diagnosing musculoskeletal conditions related to at least one body part of a subject, comprising:
monitoring at least one body part of a subject with at least one sensor, the at least one sensor configured to detect motions of the at least one body part of the subject, the at least one sensor comprising a Micro Electro Mechanical System (MEMS) sensor;
transmitting kinematic data captured when the subject moves the at least one body part in a pre-determined kinematic pattern from the at least one sensor to a signature-comparing device, the kinematic data corresponding to the motions of the at least one body part when the subject moves the at least one body part in the pre-determined kinematic pattern;
obtaining composite signatures of the subject based on the kinematic data, the composite signatures comprising at least one motion signature;
comparing the composite signatures of the subject to a normal or pre-determined established standard composite signature to determine whether a difference between the composite signatures of the subject and the normal composite signatures is larger than a pre-determined threshold; and
diagnosing, in response to determining that the difference between the composite signatures of the subject and the normal composite signatures is larger than a pre-determined threshold, the subject as having a specific musculoskeletal condition.

2. The method according to claim 1, wherein the comparing the composite signatures of the subject to the normal composite signatures to determine whether the difference between the composite signatures of the subject and the normal composite signatures is larger than the pre-determined threshold comprises:
obtaining deviations between the composite signatures of the subject and the normal composite signatures for the at least one motion signature, wherein the at least one motion signature comprises an acceleration signature, a rotation signature, a deviation of plane signature, and a timing signature;
generating a four-dimensional stick figure video based on the obtained deviations; and
analyzing the four-dimensional stick figure video to determine whether the obtained deviations is larger than the pre-determined threshold.

3. The method according to claim 1, wherein the transmitting the kinematic data by the at least one sensor to the signature-comparing device, the method further comprising:
transmitting the kinematic data by the at least one sensor to the signature-comparing device via a wireless communication.

4. The method according to claim 1, further comprising obtaining the normal composite signatures from a server by the signature-comparing device.

5. The method according to claim 1, further comprising instructing the subject to move the at least one body part in the pre-determined kinematic pattern.

6. The method according to claim 1, further comprising:
monitoring at least one body part of a plurality of normal subjects with the at least one sensor in a pre-determined configuration, the at least one sensor configured to detect motions of the at least one body part of the plurality of normal subjects;
transmitting kinematic data captured when each of the plurality of normal subjects moves the at least one body part in the pre-determined kinematic pattern from the at least one sensor to the signature-comparing device, the kinematic data of each of the plurality of normal subjects corresponding to the motions of the at least one body part of each of the plurality of normal subjects when each of the plurality of normal subjects is instructed to move the at least one body part of each of the plurality of normal subjects in the pre-determined kinematic pattern;
obtaining the normal composite signatures based on the kinematic data of each of the plurality of normal subjects, the normal composite signatures comprising at least one normal motion signature; and
transmitting the normal composite signatures by the signature-comparing device to a server.

7. The method according to claim 1, wherein the obtaining the composite signatures of the subject based on the kinematic data comprises:
analyzing the kinematic data by the signature-comparing device to generate at least one of threshold amplitude, an acceleration in a first pre-determined time period, or a velocity in a second per-determined time period as the at least one motion signature; and
analyzing the at least one motion signature to obtain the composite signatures of the subject.

8. The method according to claim 1, further comprising:
in response to a shoulder of the subject under a specific diagnosis and one of the at least one sensor attached to a groove underarm between a biceps and a triceps of the subject, monitoring with the at least one sensor the motions of the subject when the subject keeps spine erect and raises a hand at a side with an arm rotated so a thumb points towards a body of the subject with and without a weight in a hand of the subject; and
diagnosing, in response to determining that the difference between the composite signatures of the subject and the normal composite signatures is larger than the pre-determined threshold, the shoulder of subject as having the specific musculoskeletal condition with a supraspinatus injury of the subject.

9. The method according to claim 1, further comprising:
in response to an elbow of the subject under a specific diagnosis and one of the at least one sensor attached to a wrist of the subject, monitoring with the at least one sensor the motions of the subject when the subject stands and performs a throwing motion with and without a weight in a hand of the subject; and
diagnosing, in response to determining that the difference between the composite signatures of the subject and the normal composite signatures is larger than the pre-determined threshold, the elbow of subject as having the specific musculoskeletal condition with an elbow joint of the subject.

10. The method according to claim 9, wherein:
the difference between the composite signatures of the subject and the normal composite signatures comprises a restriction in motion of a z-axis.

11. The method according to claim 1, further comprising:
in response to a knee of the subject under a specific diagnosis and one of the at least one sensor attached to a tibial bone just above an ankle of the subject, monitoring with the at least one sensor the motions of the subject when the subject sits and extends the knee by lifting a foot into extension and return to a resting position with and without a weight attached to a boot on the foot of the subject; and
diagnosing, in response to determining that the difference between the composite signatures of the subject and the normal composite signatures is larger than the pre-determined threshold, the knee of subject as having the specific musculoskeletal condition.

12. The method according to claim 1, further comprising:
in response to an Achilles tendon of the subject under a specific diagnosis and one of the at least one sensor attached to a tibial bone just above an ankle of the subject, monitoring with the at least one sensor the motions of the subject when the subject stands on both feet and goes up on tip toes and return to standing position with and without a weight attached to a boot on a foot of the subject; and
diagnosing, in response to determining that the difference between the composite signatures of the subject and the normal composite signatures is larger than the pre-determined threshold, the Achilles tendon of subject as having the specific musculoskeletal condition.

13. The method according to claim 1, further comprising:
in response to a gastrocnemius muscle of the subject under a specific diagnosis and one of the at least one sensor attached to a tibial bone just above an ankle of the subject, monitoring with the at least one sensor the motions of the subject when the subject stands on both feet and bends an opposite knee and to go up on tip toes with a foot remaining on a floor and return to standing position with and without a weight attached to a boot on the foot of the subject; and
diagnosing, in response to determining that the difference between the composite signatures of the subject and the normal composite signatures is larger than the pre-determined threshold, the gastrocnemius muscle of subject as having the specific musculoskeletal condition. the subject is instructed to stand on both feet and to bend the opposite knee and to go up on the tip toes with the foot remaining on a floor and return to standing position without the weight attached to the boot on the foot of the subject.

14. The method according to claim 1, wherein:
the normal composite signatures comprise a normal x-axis acceleration signature, a normal y-axis acceleration signature, a normal z-axis acceleration signature, a normal x-axis rotation speed signature, a normal y-axis rotation speed signature, and a normal z-axis rotation speed signature; and
each of the normal composite signatures comprises a normal range corresponding to a plurality of normal subjects.

15. The method according to claim 14, wherein a normal range for each of the normal composite signatures is determined by at least one method of:
a first method to determine a range based on an average and a standard deviation,
a second method to determine a range between a low threshold and a high threshold, or
a third method to determine a range based on advanced statistics or machine learning to differentiate the normal composite signatures from abnormal composite signatures.

16. The method according to claim 15, wherein the first method is to determine a range being between the average minus three times the standard deviation and the average plus three times the standard deviation.

17. The method according to claim 15, wherein:
the low threshold is smaller than kinematic data of the plurality of normal subjects; and
the high threshold is larger than the kinematic data of the plurality of normal subjects.

18. The method according to claim 14, wherein the comparing the composite signatures of the subject to the normal composite signatures to determine whether the difference between the composite signatures of the subject and the normal composite signatures is larger than the pre-determined threshold comprises:
comparing the composite signatures of the subject to the normal composite signatures to determine whether any signature of the composite signatures is outside a normal range of a corresponding signature of the normal composite signatures.

19. A system for dynamic diagnosing musculoskeletal conditions related to at least one body part of a subject in response to a set of instructions for instructing the subject to move the at least one body part in a pre-determined kinematic pattern, the system comprising:
at least one sensor attached to the at least one body part of the subject, the at least one sensor configured to detect motions of the at least one body part of the subject; and
a signature-comparing device receiving kinematic data transmitted from the at least one sensor, the kinematic data corresponding to the motions of the at least one body part when the subject is instructed to move the at least one body part in the pre-determined kinematic pattern, so that the signature-comparing device:
obtains composite signatures of the subject based on the kinematic data, the composite signatures comprising at least one motion signature,
compares the composite signatures of the subject to normal composite signatures to determine whether a difference between the composite signatures of the subject and the normal composite signatures is larger than a pre-determined threshold, and
diagnoses, in response to determining that the difference between the composite signatures of the subject and the normal composite signatures is larger than a pre-determined threshold, the subject as having a specific musculoskeletal condition.

20. The system according to claim 19, wherein the signature-comparing device received the kinematic data transmitted from the at least one sensor via a wireless communication.

21. The system according to claim 19, wherein, when the signature-comparing device receives the kinematic data transmitted from the at least one sensor, the signature-comparing device further obtains the normal composite signatures from a server by the signature-comparing device.

22. The system according to claim 19, wherein the at least one sensor is a Micro Electro Mechanical System (MEMS) sensor.

23. The system according to claim 19, wherein, when the signature-comparing device obtains the composite signatures of the subject based on the kinematic data, the signature-comparing device:
analyzes the kinematic data to generate at least one of threshold amplitude, an acceleration in a first pre-determined time period, or a velocity in a second per-determined time period as the at least one motion signature; and
analyzes the at least one motion signature to obtain the composite signatures of the subject.

24. The system according to claim 19, wherein, when the signature-comparing device compares the composite signatures of the subject to the normal composite signatures to determine whether the difference between the composite signatures of the subject and the normal composite signatures is larger than the pre-determined threshold, the signature-comparing device:
obtains deviations between the composite signatures of the subject and the normal composite signatures for the at least one motion signature, wherein the at least one motion signature comprises an acceleration signature, a rotation signature, a deviation of plane signature, and a timing signature;
generates a four-dimensional stick figure video based on the obtained deviations; and
analyzes the four-dimensional stick figure video to determine whether the obtained deviations is larger than the pre-determined threshold.

25. The system according to claim 19, wherein:
one of the at least one sensor is attached to a groove underarm between a biceps and a triceps of the subject, wherein the subject is instructed to keep spine erect and to perform instructed motions with and without a weight in a hand of the subject; and
the signature-comparing device, in response to the difference between the composite signatures of the subject and the normal composite signatures being larger than the pre-determined threshold, provides a specific diagnosis of a shoulder of the subject as the specific musculoskeletal condition related to a supraspinatus injury of the subject.

26. A system for dynamic diagnosing musculoskeletal conditions related to at least one body part of a subject, the system comprising:
at least one sensor configured to detect motions of at least one body part of a subject, wherein the at least one sensor is attached to the at least one body part of a subject, and the subject, in response to a set of instructions, is instructed to move the at least one body part in a pre-determined kinematic pattern, wherein, when the subject moves the at least one body part in the pre-determined kinematic pattern, the at least one sensor transmits kinematic data corresponding to the motion of the at least one body part to a signature-comparing device;
a signature generating circuitry in the signature-comparing device generating composite signatures based on the kinematic data;
a comparison circuitry in the signature-comparing device comparing the composite signatures and normal composite signatures to determine whether a difference between the composite signatures of the subject and the normal composite signatures is larger than a pre-determined threshold; and
a diagnosing circuitry providing result of the subject as having a specific musculoskeletal condition in response to determining that the difference between the composite signatures of the subject and the normal composite signatures is larger than a pre-determined threshold.
